(19) United States
(12) Patent Application Publication
(10) Pub. No.: US 2005/0003380 A1
(43) Pub. Date: Jan. 6, 2005

(54) Selective gene amplification

(75) Inventors: Helen Cohen, Cambridge (GB); Bernie Kelly, Cambridge (GB); Andrew Griffiths, Cambridge (GB)

Correspondence Address: PALMER & DODGE, LLP;KATHLEEN M. WILLIAMS, 111 HUNTINGTON AVENUE, BOSTON, MA, 02199, US

(73) Assignee: Medical Research Council

(21) Appl. No.: 10/740,133

(22) Filed: Dec. 18, 2003

Related U.S. Application Data
(63) Continuation-in-part of application No. PCT/GB02/02802, filed on Jun. 18, 2002.

(30) Foreign Application Priority Data
Jun. 18, 2001 (GB) 0114854.3

(51) Int. Cl. C12Q 1/68; C12P 19/34

(52) U.S. Cl. 435/6; 435/91.2

(57) ABSTRACT

A method is provided for selecting nucleric acids encoding gene product is in which the nucleic acid encoding the gene product is selectly and quantitatively amplified depending on the activity of the gene itself or the gene product. The method may, for example, be used in selecting gene products arising from in vitro evolution of molecular libraries.

### FIELD OF THE INVENTION

The present invention relates to methods for use in selection and in vitro evolution of molecular libraries. In particular, the present invention relates to methods of selecting nucleic acids encoding gene products in which the nucleic acid encoding the gene product is selectively and quantitatively amplified depending on the activity of the gene product.

### BACKGROUND TO THE INVENTION

Evolution requires the generation of genetic diversity (diversity in nucleic acid) followed by the selection of those nucleic acids which encode beneficial characteristics. Because the nucleic acids and their encoded gene product are maintained together in biological organisms (the nucleic acids encoding the molecular blueprint of the cells in which they are confined), alterations in the genotype resulting in an adaptive change(s) of phenotype produce benefits for the organism resulting in positive selection through competitive advantage. Multiple rounds of mutation and selection can thus result in the progressive enrichment of organisms (and the encoding genotype) with increasing adaptation to a given selective condition.

The principles of Darwinian evolution can also be applied in the laboratory to generate novel proteins and nucleic acids with tailor-made binding, catalytic and regulatory activities. To do so, directed or in vitro evolution systems need to have three vital components:
1. a method of generating genetic diversity;
2. a way of linking genotype and phenotype; and
3. a selection for the desired activity allowing preferential replication of genes giving rise to the desired phenotype.

There are currently a variety of different ways to create genetic diversity, including random point mutagenesis and recombination, for examples see (Minshull & Stemmer, 1999; Ostermeier et al., 1999).

There are also currently several ways of linking genotype and phenotype in vitro and in vivo. These approaches can be classified into two major types—physical and compartmentalised—as described below.

Physical genotype-phenotype linkage. With nucleic acids, the same molecule can embody both genotype (a sequence which can be replicated) and phenotype (a functional trait such as binding or catalytic activity). This has enabled the selection, by techniques such as SELEX, of RNA and DNA molecules (aptamers) capable of binding a given ligand. Similarly, proteins with binding activities can be selected by physically linking the protein and the gene encoding it. The latter was first achieved by displaying proteins fused to coat proteins of filamentous bacteriophages (Smith & Petrenko, 1997) and later by a variety of other methods including ribosome display, mRNA-peptide fusion, plasmid-display, bacterial display and yeast display (Boder & Wittrup, 1997; Georgiou et al., 1997; Jermutus et al., 1998; Roberts, 1999; Schatz et al., 1996). In all cases, selection for binding is performed in vitro by affinity purification or fluorescence activated cell-sorting. The species retained or sorted are then amplified and cloned, or put through subsequent rounds of mutation and selection. Binding to transition-state analogues and mechanism-based-inhibitors can also be used to select (indirectly) for nucleic acids or proteins with catalytic activity (Arkin & Wells, 1998; Janda et al., 1997; Pollack et al., 1986; Tramontano et al., 1986).

Physical genotype-phenotype linkage strategies can also be used for the selection of ‘catalysts’ in an ‘intramolecular single-turnover’ mode. This strategy has been used routinely with nucleic acids (SELEX) (Roberts & Ja, 1999; Szostak & Wilson, 1999), and recently with proteins displayed on phage (Atwell & Wells, 1999; Demartis et al., 1999; Jestin et al., 1999; Pedersen et al., 1998). In principle, it could be extended to the other physical linkages described above. For these selections the substrate is linked to all nucleic acids or proteins in the library (the proteins are in turn physically linked to the gene encoding it). Subsequently the product of the reaction remains linked to the genes that encode catalysts, thus allowing their isolation via a reagent that binds the product but not the substrate. It has also proven possible to select catalytic proteins in a normal intramolecular multiple turnover mode by displaying them on the surface of bacterial cells and using a fluorogenic substrate which becomes associated with the cell surface enabling fluorescence-activated cell sorting of cells coated with the fluorescent product (Olsen et al., 2000).

Genotype-phenotype linkage by compartmentalisation. The man-made strategy of physical genotype-phenotype linkage is fundamentally different from nature's. In nature, genotype-phenotype linkage is achieved by compartmentalisation. Genes, the proteins they encode, and the products of their activity are all kept together, compartmentalised in cells. This type of linkage allows the selection of both nucleic acids and proteins. Moreover, all biological functions can be selected for, be they structural, binding, catalytic or regulatory. Moreover, in contrast to direct, physical linkage, the selected phenotype is no longer limited to the outcome of the activity of a single protein, or nucleic acid acting in isolation, but can be extended to two, or more, genes and proteins, acting together in concert, thus yielding metabolic pathways, signal transduction cascades and all other processes vital to life.

One obvious way of recruiting compartmentalisation for directed evolution is to use cells and perform the selection in vivo. In vivo selections classically complement a finction in a strain that initially lacks it (an auxotroph), neutralise a substance that is toxic or inhibits growth, or provide a substance essential for growth (Fastrez, 1997) and this approach has proven helpfil in numerous cases where this has been possible.

An in vitro selection system which uses compartmentalisation to link genotype to phenotype has also been described in WO99/02671, WO00/40712 and (Tawfik & Griffiths, 1998). Genetic elements encoding a gene product having a desired activity are compartnentalised into microcapsules (typically aqueous droplets in a water-in-oil emulsion) and then transcribed and/or translated to produce their respective gene products (RNA or protein) within the microcapsules. Genetic elements which produce gene product having desired activity are subsequently sorted. This approach selects gene products of interest by detecting the desired activity by a variety of means.

Compartmentalised self-replication is a variant of this system which also uses compartmentalisation into microcapsules formed in water-in-oil emulsions (Ghadessy et al., 2001). This approach for the directed evolution of enzymes relies on a feedback loop consisting of a polymerase that replicates only its encoding gene because the compartmentalisation isolates individual self-replication reactions from each other. Enzymes other than polymerases can be selected too if they generate products which are required for gene polymerisation.

As discussed above, a number of different methods exist for the selection and directed evolution in the laboratory of novel proteins and nucleic acids with tailor-made binding, catalytic and regulatory activities. Most of these rely on the creation of a ‘genetic element’, which contains a nucleic acid (DNA or RNA) gene and optionally a variety of other molecules. The genetic element is modified by the desired activity of the gene itself (DNA or RNA) or, after transcription, translation, or transcription and translation, by the desired activity of the RNA or protein encoded by the gene (the ‘gene product’). The selective modification of the genetic elements enables them to be separated from unmodified genetic elements (or at least selectively enriched).

Many of the methods for in vitro selection and evolution are based on creating a physical genotype-phenotype linkage. Where the phenotypic trait is exerted directly by the nucleic acid, the genetic element may simply be the DNA or RNA since the same molecule can embody both genotype (a sequence which can be replicated) and phenotype (a functional trait such as binding or catalytic activity). Where the phenotype is exerted by proteins, the gene (DNA or RNA) and the protein encoded by the gene can be physically linked in a variety of ways, including phage display, ribosome display, mRNA-peptide fusion, plasmid-display, bacterial display and yeast display (Boder & Wittrup, 1997; Georgiou et al., 1997; Jermutus et al., 1998; Roberts, 1999; Schatz et al., 1996). These systems can be used to select for binding activities: the genetic elements containing binding proteins are selectively modified as a result of binding the target ligand which enables purification of the genetic element by, for example, affinity purification or fluorescence-activated cell sorting.

Genetic elements modified as a result of an activity (binding, catalytic, or regulatory) of a nucleic acid or protein may be selected in an in vitro system which uses compartmentalisation to link genotype to phenotype as described in WO99/02671, WO00/40712 and (Tawfik & Griffiths, 1998).

### SUMMARY OF THE INVENTION

The present invention provides an improved means of selecting genes having or encoding a desired activity using a compartmentalised in vitro selection system. Specifically, this invention provides a novel way of specifically replicating genes in genetic elements modified as a result of a desired activity, thereby enriching for these genes. A particular advantage is that the degree of enrichment of a gene is directly proportional to the degree of modification of the genetic element containing the gene and hence closely reflects the level of activity of the gene or gene product in the genetic element. This is achieved first by the formation of a genetic element, for example by any of the methods described above. The genetic element comprises a gene (a nucleic acid molecule) and optionally a variety of other molecules which may be modified, directly or indirectly, by the action of the gene or gene product, which may be a structural, binding, catalytic, regulatory or other action. For example, the genetic element may comprise (in addition to the gene) one or more substrates which may be converted to product, modified or otherwise altered by the action of a gene product. The degree of amplification of the gene comprised by a given genetic element (i.e. the number of new copies of the gene) is therefore related to the degree of modification of that genetic element. For example, in the case of a genetic element comprising a substrate molecule which is converted to product (which remains a component of the genetic element) by the action of the gene itself or by the action of the gene product, the gene contained in that genetic element is amplified to a degree related to the amount of product molecule formed. This method links the fitness of a gene, measured as its ability to be replicated, directly to the activity, e.g. catalysis of product formation, of the gene product it encodes. In other words, the number of copies of a given gene in the gene population after selection would be proportional to the activity of the encoded gene product. Such a selection method is more advantageous than alternative in vitro selection strategies, as it more closely mimics, at the molecular level, the powerful process of natural selection of organisms.

The technique according to the invention is especially useful for gene products displaying low turnover as well as in methods for selecting enzymes for a novel catalytic finction, when it is likely that the first generation of mutants show low levels of activity at best.

Accordingly, in a first aspect the present invention provides a method for selecting one or more genetic elements encoding a gene product having a desired activity, which method comprises:
(i) providing a plurality of genetic elements comprising a nucleic acid optionally encoding a gene product;
(ii) optionally expressing the nucleic acids to produce the gene products, and allowing the desired activity of the nucleic acids or gene products to result, directly or indirectly, in the modification of the genetic elements which contained or encoded them;
(iii) associating one or more modulators of a nucleic acid replication system with a genetic element which has been modified by a gene product;
(iv) selectively amplifiing the nucleic acid component of those genetic elements which have been modified by the gene product.

In the context of the invention, the gene-product may be encoded by some or all of the nucleic acids within the genetic element. Typically, the nucleic acids encode a repertoire of gene product molecules in which one or more possess an activity which renders them desirable. The procedure is configured to select such nucleic acids, by assaying for the desired activity of the gene or the gene products that they encode.

In particular, the present invention provides a method for selectively amplifying nucleic acid sequences from a plurality of genetic elements encoding gene products, which method comprises
(i) providing a plurality of genetic elements comprising a nucleic acid optionally encoding a gene product;
(ii) optionally expressing the nucleic acids to produce the gene products, and allowing the desired activity of the nucleic acids or gene products to result, directly or indirectly, in the modification of the genetic elements which contained or encoded them;
(iii) selectively attaching to the modified genetic element at least a first component which potentiates amplification of the nucleic acid molecules;
(iv) dividing the plurality of genetic elements into a number of separate compartments,
(v) providing in each compartment further components for nucleic acid amplification such that only nucleic acid molecules linked to modified. genetic elements to which said first component has been attached will be amplified; and
(vi) allowing amplification to occur in the compartments of the nucleic acid component of those genetic elements to which said first component has been attached.

In an alternative embodiment, the invention provides a method for selectively amplifying nucleic acid sequences from a plurality of genetic elements encoding gene products, which method comprises

(i) providing a plurality of genetic elements comprising a nucleic acid optionally encoding a gene product;

(ii) optionally expressing the nucleic acids to produce the gene products, and allowing the desired activity of the nucleic acids or gene products to result, directly or indirectly, in the modification of the genetic elements which contained or encoded them;

(iii) selectively attaching to unmodified components of genetic elements at least a first component detrimental to the amplification of the nucleic acid molecules comprised by the genetic elements;

(iv) dividing the plurality of genetic elements into a number of separate compartments,

(v) providing in each compartment further components for nucleic acid amplification; and

(vi) allowing nucleic acid amplification to occur in the compartments.

In one embodiment, the firther components for nucleic acid amplification are selected from components for polymerase chain reaction (PCR) (Saiki et al., 1988), or a variant thereof. Alternative amplification systems may be exploited, including ligase chain reaction (LCR) (Wu & Wallace, 1989) strand displacement amplification (SDA) (Walker et al., 1992a), and nucleic acid sequence-based amplification (NASBA) (Compton, 1991) or self-sustaining sequence replication (3SR) (Guatelli et al., 1990), transcription-mediated amplification (IMA) (U.S. Pat. No. 5,399,491) and rolling circle amplification (RCA) (Lizardi et al., 1998).

The first component or modulator of the nucleic acid replication system, which potentiates or is detrimental to amplification of the nucleic acid molecules, may for example be a component of the nucleic acid replication system itself, such as a primer or an enzyme or cofactor therefor. Where the component is detrimental to the replication, it is advantageously a competitor for a component of the amplification system, such as a dideoxy-terminated primer, or a competitor for the nucleic acid molecule.

In the context of the present invention, it will be understood that amplification requires replication of nucleic acids.

Preferably, the first component comprises or is otherwise attached to a moiety capable of binding to modified genetic elements but not unmodified genetic elements.

In accordance with the invention, migration of the component of the replication system from modified to unmodified genetic elements should be substantially prevented. This may be achieved in a number of ways. For example, in cases where the component, for example a primer, must be detached from the genetic element in order to function in the replication system, the genetic elements and components may be placed in a difflusion-limiting medium, such as a gel; alternatively, they may be compartmentalised, for example using microcapsules such as emulsion vesicles as described in more detail herein.

Alternatively, the component may be tethered to the genetic element such that is only able effectively to interact with the element to which it is tethered. The use of linkers to attach primers to a solid phase is known for example in bridge amplification (U.S. Pat. No. 5,641,658). Preferably, the linkers are flexible.

In a highly preferred embodiment, the genetic elements are compartmentalised. Compartmentalisation is preferably achieved by the use of water-in-oil emulsions i.e. step (iv) of the method of the invention described above comprises forming a water-in-oil emulsion of an aqueous solution comprising the genetic elements, bound first component and further components for amplification in an oil-based medium.

Preferably the plurality of nucleic acid molecules is, or is obtained from, a library of nucleotide sequences encoding a gene product and variants thereof.

Optionally, the method of the invention further comprises recovering one or more of the amplified nucleic acid molecules and determining all or part of their nucleotide sequence.

In a further aspect, the present invention provides a kit that may be used in the above methods, the kit comprising:
(i) a plurality of genetic elements optionally encoding a plurality of gene products, each genetic element comprising a nucleic acid sequence optionally operably linked to a regulatory sequence which is capable of directing expression of a gene product encoded by the sequence, wherein the desired activity of the gene or gene products results, directly or indirectly, in the modification of the genetic elements which contained/encoded them;
(ii) a first component which modulates amplification of the nucleic acid constructs which is capable of selectively binding to genetic elements modified by the action of the gene or gene products but not to unmodified genetic elements.

Since the present invention is concerned with selecting gene products with improved or novel properties, in another aspect, the present invention also provides a method of selecting a nucleic acid encoding a gene product having a desired activity, which method comprises subjecting a plurality of nucleic acid molecules encoding a plurality of gene products to selective amplification by the above method of the invention and selecting one or more nucleic acid molecule which are amplified to a desired extent. Also provided are gene products selected by this method.

In another embodiment, the present invention provides a method of selecting a variant of a gene which variant has altered activity compared with the original gene, which method comprises subjecting a plurality of nucleic acid molecules and variants thereof to selective amplification by an above method of the invention and selecting a nucleic acid which is amplified to a different extent to the original gene. Also provided are genes selected by this method.

In another embodiment, the present invention provides a method of selecting a variant of a gene product which variant has altered activity compared with the original gene product, which method comprises subjecting a plurality of nucleic acid molecules encoding the gene product and variants thereof to selective amplification by an above method of the invention and selecting a nucleic acid molecule encoding a variant of the gene product which is amplified to a different extent to a nucleic acid molecule encoding the original gene product. Also provided are genes and gene product variants selected by this method.

According to a frther aspect of the present invention there is provided a method of in vitro evolution comprising the steps of:
(a) selecting one or more nucleic acids from a library of nucleic acids according to the present invention;
(b) mutating and/or recombining the selected nucleic acid(s) in order to generate a further library of nucleic acids, optionally encoding a repertoire of gene products;
(c) iteratively repeating steps (a) and (b) in order to obtain a gene or gene product with enhanced activity.

In a preferred embodiment, the genetic elements, conditionally modified depending on the activity of the gene or gene product, are created using the methods described in WO99/02671, WO00/40712 and (Tawfik & Griffiths, 1998). This is an in vitro system which uses compartmentalisation to link genotype to phenotype. Genetic elements are compartmentalised into microcapsules (typically aqueous droplets in a water-in-oil emulsion) and then transcribed and/or translated to produce their respective gene products (RNA or protein) within the microcapsules. Genetic elements which produce gene product having desired activity become modified within the microcapsules. In a highly preferred embodiment, the genetic element comprises the gene attached to a solid phase, such as a microsphere, and optionally a variety of other molecules.

### BRIEF DESCRIPTION OF THE FIGURES

FIG. 1—Emulsion—PCR—selection scheme.

FIG. 2—Asymmetric PCR—Graph of amount of limiting primer extended at various concentrations.

FIG. 3—Scheme showing attachment of biotinylated primers to product immobilised on microspheres via an avidin bridge and an anti-product antibody.

FIG. 4—Picture of gel showing results of PCR with one primer imrnobilised on microspheres.

FIG. 5—Picture of gel showing results of PCR in an emulsion, with various concentrations of BSA.

FIG. 6—Picture of gel showing (A) emulsion-PCR reaction product, after 2nd round of (nesting) amplification (B) solution PCR reaction products, after 2nd round of (nesting) amplification.

FIG. 7—Graph of degree of amplification of gene in an emulsion versus number of primer molecules.

FIG. 8—Picture of gel showing estimation by competitive PCR of gene amplification with primers recruited to product molecules on microspheres.

FIG. 9—Picture of gel showing linkage of oligonucleotide to antibody.

FIG. 10—Picture of gel showing enrichment of N-FLAG-OPD-HA gene from excess of DHFR-HA gene.

### DETAILED DESCRIPTION OF THE INVENTION

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2^{nd} ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology, which are incorporated herein by reference) and chemical methods.

Selection is the mechanism of evolution, and with powerfil new techniques for applying selection pressure to genes and proteins in vitro and in vivo, biologists have developed a range of procedures for sieving libraries of proteins (whether natural or engineered) for enzymes and other useful molecules. Such selection methods provide two extremely useful new abilities. Firstly, protein engineers have begun to evolve new function from existing protein scaffolds by mimicking the process of natural evolution; and secondly, biologists are developing rapid methods for isolating natural proteins from genomic and cDNA libraries on the basis of their activity.

The “ideal” selection system may be characterised as comprising the following features:
1. It allows the efficient selection of proteins with low turnover;
2. It can discriminate between small differences in turnover, allowing, for example, the evolution of a catalytic activity by gradual improvements;
3. High product turnover can also be efficiently selected, for example in cases where one desires an enzyme able to convert a large amount of substrate into product in a short space of time.

With all these features, the ideal selection system would in principle be able to evolve, for example, an enzyme which catalyses a novel transformation, starting from a protein displaying low activity and improving its performance until the desired level of activity is reached.

The invention provides a novel technique whereby genes are selectively amplified by an amount which is directly proportional to the activity of the gene product.

A number of different methods exist for the selection and directed evolution in the laboratory of novel proteins and nucleic acids with tailor-made binding, catalytic and regulatory activities. Most of these rely on the creation of a ‘genetic element’, which contains a nucleic acid (DNA or RNA) gene and optionally a variety of other molecules. The genetic element is modified by the desired activity of the gene itself (DNA or RNA) or, after transcription, translation, or transcription and translation, by the desired activity of the RNA or protein encoded by the gene (the ‘gene product’). The selective modification of the genetic elements enables them to be separated from unmodified genetic elements (or at least selectively enriched).

Many of the methods for in vitro selection and evolution are based on creating a physical genotype-phenotype linkage. With nucleic acids, the genetic element may simply be the DNA or RNA since the same molecule can embody both genotype (a sequence which can be replicated) and phenotype (a functional trait such as binding or catalytic activity). With proteins, the gene (DNA or RNA) and the protein encoded by the gene can be physically linked in a variety of ways, including phage display, ribosome display, mRNA-peptide fusion, plasmid-display, bacterial display and yeast display (Boder & Wittrup, 1997; Georgiou et al., 1997; Jermutus et al., 1998; Roberts, 1999; Schatz et al., 1996; Smith & Petrenko, 1997). These systems can be used to select for binding activities: the genetic elements containing binding proteins are selectively modified as a result of binding the target ligand which enables purification of the genetic element by, for example, affinity purification or fluorescence-activated cell sorting (FACS).

As used herein, a genetic element is a molecule or molecular construct comprising a nucleic acid. The genetic elements of the present invention may comprise any nucleic acid (for example, DNA, RNA or any analogue, natural or artificial, thereof). The nucleic acid component of the genetic element may moreover be linked, covalently or non-covalently, to one or more molecules or structures, including proteins, chemical entities and groups, solid-phase supports such as magnetic beads, and the like. In the method of the invention, these structures or molecules can be designed to assist in the sorting and/or isolation of the genetic element encoding a gene product with the desired activity.

Genetic elements can also be used to select (indirectly) catalytic nucleic acids and proteins by binding to transition-state analogues (TSAs) or mechanism-based inhibitors (Arkin & Wells, 1998; Janda et al., 1997; Pollack et al., 1986; Tramontano et al., 1986). Genetic elements containing ‘catalytic’ nucleic acids or proteins can also be selected in an ‘intramolecular single-turnover’ mode or an intramolecular multiple turnover mode as described above. In this case the genetic elements which contain ‘catalytic’ nucleic acids or proteins become modified by generation of the product of the ‘catalysed’ reaction which is, or becomes, a component of the genetic element.

In most of these cases enrichment of genes in genetic elements modified as a result of the desired activity is performed by affinity purification or FACS.

The present invention provides an improved means of selecting genes having or encoding a desired activity using a compartmentalised in vitro selection system. Specifically, this invention provides a novel way of specifically replicating genes in genetic elements modified as a result of a desired activity, thereby enriching for these genes. A particular advantage is that the degree of enrichment of a gene is directly proportional to the degree of modification of the genetic element containing the gene and hence closely reflects the level of activity of the gene or gene product in the genetic element. This is achieved first by the formation of a genetic element, for example, by any of the methods described above. The genetic element comprises a gene (a nucleic acid molecule) and optionally a variety of other molecules which may be modified, directly or indirectly, by the action of the gene or gene product, which may be a structural, binding, catalytic, regulatory or other action. For example, the genetic element may comprise (in addition to the gene) substrate which may be converted to product by the action of a gene product. The degree of amplification of the gene (i.e. the number of new copies of the gene) comprised by a given genetic element is then related to the degree of modification of that genetic element. For example, in the case of a genetic element comprising a substrate molecule which is converted to product (which remains a component of the genetic element) by the action of the gene itself or by the action of the gene product, the gene contained in that genetic element is amplified to a degree related to the amount of product molecule formed. This method links the fitness of a gene, measured as its ability to be replicated, directly to the activity, e.g. catalysis of product formation, of the gene product it encodes. In other words, the number of copies of a given gene in the gene population after selection would be proportional to the activity of the encoded gene product. Such a selection method is more advantageous than alternative in vitro selection strategies, as it more closely mimics, at the molecular level, the powerful process of natural selection of organisms.

### Creation of Genetic Elements

A variety of methods exist for the formation of genetic elements. In all cases the genetic element contains at least a nucleic acid molecule.

Nucleic acid molecules for use in the methods of the present invention are typically DNA or RNA. They may be single stranded or double stranded. Single stranded molecules may comprise double-stranded regions, for example due to intramolecular base-pairing. Nucleic acid molecules are typically linear or circular.

The nucleic acid molecules optionally comprise a sequence which encodes a gene product in which case the sequence will typically include the actual coding sequence operably linked to a regulatory control sequence that is capable of providing for the expression of the coding sequence in an appropriate expression system. The term “operably linked” means that the components described are in a relationship permitting them to fimction in their intended manner. A regulatory sequence “operably linked” to a coding sequence is ligated in such a way that expression of the coding sequence is achieved under conditions compatible with the control sequences. The nucleic acid molecule may be for example, part of a plasmid, phage or virus vector.

Regulatory control sequences include promoters/enhancers and other expression regulation signals such as transcription terminators and translational control sequences which function in transcription/translation systems. These control sequences are generally selected to be compatible with the expression system in which the nucleic acid molecule is intended to be used. The term “promoter” is well-known in the art and encompasses nucleic acid regions ranging in size and complexity from minimal promoters to promoters including upstream elements and enhancers.

Nucleotide sequences to be screened according to the present invention typically either have an activity of interest or encode RNA molecules or polypeptides having an activity of interest The activity may be catalytic, binding, regulatory or any other activity.

Polypeptides having an activity of interest include antibodies, antigens, enzymes, ligands such as growth factors, receptors such as cell surface receptors, other components of cellular signal transduction pathways, nucleic acid binding proteins such as nucleic acid repair enzymes, polymerases, recombinases and transcription factors, and structural proteins. Polypeptides also include fragments of the above and fusion constructs encoding fragments from different proteins in a single polypeptide.

The nucleic acids or polypeptides may be non-randomised, for example ‘wild-type’ or allelic variants of naturally occurring nucleic acids or polypeptides, or may be specific mutant(s), or may be wholly or partially randomised. Thus, the nucleic acid molecules are typically provided as a plurality of nucleic acid molecules, each optionally encoding a different gene product such as a member of a randomised library of sequences. Preferably, there are at least 50, 100, 500 or 1000 different sequences in the library. The library of nucleic acid molecules advantageously encodes a repertoire of gene products.

A repertoire is a population of diverse variants, for example polypeptide variants which differ in amino acid sequence. Differences in amino acid sequence are typically introduced at the DNA level by nucleic acid mutagenesis.

Randomisation is accomplished at the nucleotide level by any suitable means of mutagenesis. Mutagenesis may be performed, for example, by synthesising novel genes optionally encoding mutant gene products and optionally expressing these to obtain a variety of different gene products. Alternatively, existing genes can themselves be mutated, such as by site-directed or random mutagenesis, to obtain the desired mutant genes.

Mutations may be introduced by any method known to those of skill in the art. A number of site-directed mutagenesis methods are known in the art, from methods employing single-stranded phage such as M13 to PCR-based techniques (see.(Innis et al., 1990)).

Pluralities of nucleotide sequences encoding gene products of interest may be obtained from cloned genomic DNA or cDNA. Libraries of genes can also be made which encode all (see for example (Parmley & Smith, 1988)) or part of genes (see for example (Lowman et al., 1991))or pools of genes (see for example (Nissim et al., 1994))

Pluralities of nucleotide sequences can also be made by introducing mutations into a nucleotide sequence or pool of nucleotide sequences ‘randomly’ by a variety of techniques in vivo, including; using ‘mutator strains’, of bacteria such as E. coli mutD5 (Low et al., 1996); and using the antibody hypennutation system of B-lymphocytes (Yelamos et al., 1995). Random mutations can also be introduced both in vivo and in vitro by chemical mutagens, and ionising or UV irradiation (Friedberg et al., 1995), or incorporation of mutagenic base analogues (Zaccolo et al., 1996). ‘Random’ mutations can also be introduced into genes in vitro during polymerisation for example by using error-prone polymerases (Leung et al., 1989).

Further diversification can be introduced by using homologous recombination either in vivo (Kowalczykowski et al., 1994) or in vitro (Stemmer, 1994a; Stemmer, 1994b; Zhao et al., 1998) or non-homologous recombination (Ostermeier et al., 1999).

A further library of nucleotide sequences for use in the present invention is a PCR-assembled combinatorial library. In this case the DNA fragments of the library are assembled into full-size constructs in vitro by PCR, for example by overlap extension (Ho et al., 1989; Horton et al., 1989) or by ligase chain reaction (Barany, 1991; Landegren et al., 1988);(U.S. Pat. No. 4,883,750).

Theoretical and practical studies indicate that the larger the number of nucleic acid molecule variants screened in a combinatorial library the more likely it is that a molecule will be created with the properties desired (Lancet et al., 1993; Perelson & Oster, 1979; Varga et al., 1991). Thus, to ensure that rare variants are generated and thus are capable of being selected, a large library size is desirable. On the other hand, there is no such requirement in the case of cDNA libraries. In a perfectly normalised S. cerevisiae cDNA library full diversity could be sampled with an aliquot containing only about 6000 genes whilst H. sapiens cDNA library representing at least one transcript from each gene would require only about 30000-40000 genes. In practice allowances must be made for the probability of statistical representation of the genes, unless the source is fully arrayed and annotated.

Overall, the plurality of nucleic acid molecules preferably comprises at least 50, 100, 500 or 1000 different molecules.

Each nucleic acid molecule forms part of a genetic element which may be created in a variety of different ways, including, but not exclusively, SELEX (Roberts & Ja, 1999; Szostak & Wilson, 1999), phage display (Smith & Petrenko, 1997), ribosome display, mRNA-peptide fusion, plasmid-display, bacterial display and yeast display (Boder & Wittrup, 1997; Georgiou et al., 1997; Jermutus et al., 1998; Roberts, 1999; Schatz et al., 1996) or microencapsulation (WO99/02671, WO00/40712 and (Tawfik & Griffiths, 1998)). Creation of the genetic element may involve the expression of a gene product.

SELEX

Nucleic acid molecules may themselves posses an activity which is desirable; in such cases, the genetic element comprises the nucleic acid molecule (DNA or RNA) and, optionally, a variety of other molecules. For example, a substrate, may be linked to the nucleic acid to allow selection for ‘catalysis’ (or, more accurately, for product formation in an intramolecular single-turnover reaction) (Roberts & Ja, 1999; Szostak & Wilson, 1999).

Phage Display

The genetic element may be formed by displaying the gene product fused to coat proteins of filamentous bacteriophages (Smith & Petrenko, 1997). Optionally, further molecules, such as a substrate, may be linked to the phage particles so formed (Atwell & Wells, 1999; Demartis et al., 1999; Jestin et al., 1999; Pedersen et al., 1998).

Ribosome Display

The genetic element may be formed in an in vitro translation system by stalling the ribosome as it completes the translation of an mRNA, typically by including no stop codons at the 3′ end. The nascent peptide chain protrudes from the ribosome, forming a physical linkage between the gene product and the gene; the genetic element comprises the peptide-gene complex so formed (Mattheakis et al., 1994).

RNA-Peptide Fusion

Peptides are translated from an mRNA-DNA-puromycin conjugate. When the ribosome reaches the RNA-DNA junction, it stalls briefly, and the puromycin is able to enter the peptidyl transferase centre, where it forms a covalent linkage with the nascent peptide chain. The result is a covalent linkage between the gene product (the peptide) and the gene (the mRNA); the genetic element comprises this complex (Roberts & Szostak, 1997).

Plasmid Display

Plasmids express random peptides fused to individual DNA-binding proteins in vivo within a cell; the fusion proteins bind specifically to a sequence present in the plasmid, thus creating a physical linkage between the gene (the plasmid) and the gene product (the fuision protein). The genetic elements comprise these plasmid-fusion protein complexes (Schatz et al., 1996).

Cell Surface Display

Genetic elements may be formed by expressing genes within living cells including, but not limited to, E. coli or yeast cells, in such a way that the gene products become displayed on the cell surface. The genetic element then comprises the cell, which contains the gene, and the surface-displayed gene product, and optionally a variety of other molecules which may be attached to the cell surface (Georgiou et al., 1997).

In Vitro Compartmentalisation

In a preferred embodiment, the genetic elements are formed within microcapsules, as described in WO99/02671, WO00/40712 and (Tawfik & Griffiths, 1998). Optionally, the genetic elements so formed comprise the gene product linked physically to the gene.

Modification Of Genetic Elements

Genetic elements may be modified in a number of ways which facilitate the isolation and/or selection of those genetic elements having or encoding a desired activity. Such methods include but are not restricted to the following.

Physical Genotype-Phenotype Linkages

In those cases where the genetic element contains a physical linkage between the gene and the gene product, or in which the gene itself may have the desired activity, the genetic element may be modified in such a way that those genetic elements having or encoding a desired activity may be isolated or selected. Such methods include but are not restricted to the following:

Ligand Binding

If the desired activity is a binding activity, the genetic elements may be mixed with a ligand so that those genetic elements having or encoding the desired binding activity become modified by the binding of the ligand, whereas those genetic elements which do not have or encode the desired binding activity do not become so modified.

Binding to a Transition-State Analogue (TSA) or Mechanisnm-Based Inhibitor

In some cases it is possible to select indirectly for a desired catalytic activity by selecting genes or gene products which bind to an analogue of the transition state of the reaction for which the catalyst is sought, or by selecting genes or gene products which bind to a mechanism-based inhibitor. In such cases, the genetic elements may be mixed with the TSA or mechanism-based inhibitor, so that those genetic elements having or encoding binding activity towards the TSA or mechanism-based inhibitor become modified by the binding of the TSA or mechanism-based inhibitor.

Intramolecular Catalysis or Single-Turnover Product Formation

In some cases it is possible to select genes or gene products having a desired catalytic activity by modifying the genetic element with a substrate for the reaction to be catalysed. Genes or gene products within the genetic elements having or encoding the desired catalytic activity may catalyse the transformation of the attached substrate into product. Similarly, single-turnover product formation may be selected thus.

Intermolecular Catalysis

Genetic elements comprising, for example, cell surface-displayed gene products may be further modified by the attachment of substrate molecules to the same cell surface. Gene products having the desired catalytic activity may catalyse the transformation of some or all of the attached substrate molecules, thus modifying the genetic elements encoding the gene products having the desired activity by transformation of the attached substrate molecules.

In Vitro Compartrnentalisation

A preferred method of modifying the genetic elements is to compartmentalise them using the methods described in WO99/02671, WO00/40712 and (Tawfik & Griffiths, 1998). Methods by which the genetic elements may be formed include, but are not restricted to, the methods described above (Physical genotype-phenotype linkages). In a preferred embodiment, the genetic elements are transcribed, or transcribed and translated, compartmentalised within microcapsules, and the genetic element(s) within the same microcapsule modified as a result, direct or indirect, of the activity of the gene product using the methods described in WO99/02671, WO00/40712 and (Tawfik & Griffiths, 1998).

If the desired activity is a binding activity, the gene product may be constitutively linked to the genetic element, so that ligand may bind to the genetic element via a specific interaction; alternatively, the ligand may be linked physically to the genetic element, so that the gene product becomes associated with the genetic element only if the gene product has the desired binding activity, in which case the gene product contains a ‘tag’, such as an epitope, which may be bound specifically by a molecule, such as an antibody.

If the desired activity is a catalytic activity (or is coupled to a catalytic activity), the substrate may be linked to the genetic element so that those genetic elements having or encoding the desired activity become modified by the transformation of the substrate into product. Alternatively, the substrate molecule may be present in solution, and may subsequently become associated with the genetic element after conversion to product. For example, the substrate molecule may contain a moiety which may bind to the genetic element under certain inducible conditions (e.g. of pH), or it may contain a protected moiety which binds to the genetic element once deprotected (e.g. a caged biotin group (Pirrung & Huang, 1996; Sundberg et al., 1995)). Within compartments which contain a genetic element encoding the desired activity, some or all of the substrate molecules may become transformed into product and subsequently both the remaining substrate molecules and the product molecules may become associated with the genetic element. Alternatively, product molecules formed as a result, direct or indirect, of the activity of the gene product, may become specifically bound to the genetic element, for example by means of a specific anti-product antibody. In such a case, the substrate (and the product) may contain a moiety which may be bound specifically by a molecule such as an antibody, but only product molecules become associated with the genetic element.

In a further embodiment, genetic elements comprising the gene product physically linked to the gene, and optionally a variety of other molecules, may be formed within compartments and the genetic elements so formed recompartmentalised into further compartments in which the activity of the gene product results, directly or indirectly, in the modification of the genetic element.

Modification to the genetic element which is indirectly the result of the activity of the gene or gene product may be achieved by coupling a first reaction to subsequent reactions that take place in the same compartment, as described in WO99/02671.

In a further embodiment the methods described in WO99/02671 and WO00/40712 are used to create modified genetic elements conditionally as a result of a property of the gene itself, for example to select for regulatory control sequences, including promoters/enhancers and other expression regulation signals such as transcription terminators and translational control sequences which function in transcription/translation systems.

Gene Amplification

The present invention provides a method for selectively amplifying genes within genetic elements which have been modified. The modified genetic elements may be formed in a variety of ways, including, but not restricted to, those described above. One or more components of a nucleic acid replication system are recruited to modified genetic elements but not unmodified genetic elements, and the genetic elements may be subsequently divided into a number of separate compartments. The genes contained within modified genetic elements are amplified using a nucleic acid replication system within the compartments.

Amplification

“Amplification” refers to the increase in the number of copies of a particular nucleic acid fragment (or a portion of this). This amplification may result from an enzymatic chain reaction (such as a polymerase chain reaction (PCR), or a variant thereof, a ligase chain reaction (LCR), a strand displacement amplification (SDA), and a nucleic acid sequence-based amplification (NASBA) or a self-sustaining sequence replication (3SR)). Preferably, the amplification according to the invention is an exponential amplification, as exhibited by for example the polymerase chain reaction.

Many target and signal amplification methods have been described in the literature, for example, general reviews of these methods in(Landegren et al., 1988; Schweitzer & Kingsmore, 2001). These amplification methods may be used in the methods of our invention, and include the polymerase chain reaction (PCR), or a variant thereof, ligase chain reaction (LCR), strand displacement amplification (SDA), and nucleic acid sequence-based amplification (NASBA) or self-sustaining sequence replication (3SR)).

Polymerase Chain Reaction (PCR)

PCR is a nucleic acid amplification method described in U.S. Pat. Nos. 4,683,195 and 4,683,202. PCR consists of repeated cycles of DNA polymerase generated primer extension reactions. The target DNA is heat denatured and two oligonucleotides, which bracket the target sequence on opposite strands of the DNA to be amplified, are hybridised. These oligonucleotides become primers for use with DNA polymerase. The DNA is copied by primer extension to make a second copy of both strands. By repeating the cycle of heat denaturation, primer hybridisation and extension, the target DNA can be amplified a million fold or more in about two to four hours. An advantage of PCR is that it amplifies the amount of target DNA by 1 million to 1 billion fold in approximately 4 hours.

Each compartment in the amplification comprises components for a PCR reaction, for example, deoxynucleoside triphosphates (dNTPs), buffer, magnesium, and oligonucleotide primers. However, compartments which lack a modified substrate will lack one of the PCR components. This may be a primer or a polymerase or other component. Where the first component is a primer, one of the primers required for the PCR process will be a limiting reagent and the extent of amplification will depend mainly on the number of primers in a compartment. This is termed asymmetric PCR (is et al., 1990) and is a preferred amplification technique according to the method of the present invention. When an oligonucleotide primer is the first component, as in this format, it may be advantageous to remove subsequently any single-stranded nucleic acid product formed by extension of the second primer.

In compartments where the first component is present, all components required for the PCR process will be present and therefore amplification can occur. For example, where the gene product is an enzyme, the number of molecules of product and therefore the number of first components will depend on the activity of the gene product in the earlier stages of the selection method. In particular, where the first component is a primer, the PCR reaction is termed asymmetric PCR. Thus the extent of amplification will vary depending on the amount of first component which in turn depends on the activity of the gene product.

Further amplification may be achieved by the use of a subsequent symmetrical PCR using both primers at equal concentrations. Preferably the primers for subsequent amplification should sit prime on nucleic acid sequences lying inside the first primer (nested PCR, as demonstrated in the Examples). Other amplification techniques may also be used.

Reverse Transcriptase-PCR

RT-PCR is used to amplify RNA targets (Wang et al., 1989). In this process, the reverse transcriptase enzyme is used to convert RNA to complementary DNA (cDNA), which can then be amplified using PCR. This method has proven useful for the detection of RNA viruses.

The methods of the invention may employ RT-PCR in the amplification step. Thus, the pool of nucleic acids encoding the replicase or its variants may be provided in the form of an RNA library. Such a library may be generated in vivo in bacteria, mammalian cells, yeast etc which are compartmentalised, or by in-vitro transcription of compartmentalised DNA. Other components necessary for amplification (polymerase and/or reverse transcriptase, dNTPs, primers) are also compartmentalised as described above for PCR. Only in compartments where a first component of the RT-PCR components is present will amplification take place.

Nucleic Acid Sequence-Based Amplification (NASBA) or Self-Sustained Sequence Replication (3SR)

Self-sustained sequence replication (3SR or NASBA) involves the isothermal amplification of a nucleic acid template via sequential rounds of reverse transcriptase (RT), polymerase and nuclease activities that are mediated by an enzyme cocktail and appropriate oligonucleotide primers (Guatelli et al., 1990). Enzymatic degradation of the RNA of the RNA/DNA heteroduplex is used instead of heat denaturation. RNase H and all other enzymes are added to the reaction and all steps occur at the same temperature and without further reagent additions. Following this process, amplifications of 10⁶ to 10⁹ have been achieved in one hour at 42 degrees C.

The methods of the invention may therefore use 3SR isothermal amplification (Guatelli et al., 1990) as the amplification step instead of PCR thermocycling. As described above, 3SR involves the concerted action of two enzymes: an RNA polymerase as well as a reverse transcriptase cooperate in a coupled reaction of transcription and reverse transcription, leading to the simultaneous amplification of both RNA and DNA.

Ligase Chain Reaction (ECR)

The ligase chain reaction or ligation amplification system uses DNA ligase and four oligonucleotides, two per target strand (Wu & Wallace, 1989). The oligonucleotides hybridise to adjacent sequences on the target DNA and are joined by the ligase. The reaction is heat denatured and the cycle repeated.

Alternative Amplifcation Methods

The invention moreover comprises the use of any amplification technique which is available to those skilled in the art. Such techniques include, but are not limited to, rolling circle amplification (Lizardi et al., 1998) and strand-displacement amplification (SDA) (Walker et al., 1992b). These disclosures are incorporated herein by reference in their entirety.

Modification of the protocols set forth above may be advantageous in the context of emulsion amplification of nucleic acids, and is envisaged within the scope of the present invention. For example, the inventors have observed that the addition of BSA to a PCR amplification mixture enhances the yield of the amplification. The invention thus includes the use of proteins, nucleic acids and other compounds to increase the performance of amplification reactions in emulsions.

Modulators of Nucleic Acid Replication Systems

As used herein, “modulation” refers to the increase or decrease in the activity of a nucleic acid replication system such that nucleic acid is replicated to a greater or lesser extent Preferably, the activity of the replication system is modulated in respect of a specific nucleic acid, which is advantageously the nucleic acid comprised in the genetic element.

The modulator may potentiate the replication reaction; for example, the modulator can result in an increase of 10%, 20%, 30%, 40%, 50% or more in the rate of replication. Advantageously, it is an increase of 100%, 200%, or more. Likewise, modulators detrimental to the replication reaction can decrease the rate of replication by a similar amount.

In a broad embodiment, the modulator of the nucleic acid replication system is a component thereof, required for the functioning of the system, or an agent capable of increasing or decreasing the activity of a component of the replication system. For example, the modulator may be a primer for the nucleic acid, which permits the nucleic acid amplification reaction to take place. Nucleic acid replication systems typically comprise several components, one or more of which may be suitable for recruitment to modified genetic elements. In a preferred embodiment, the number of copies of the component recruited to a given genetic element limits the replication of the gene contained within the genetic element, so that the number of new copies of a gene replicated is related to the degree of modification of the genetic element.

Examples of components which may be suitable for recruitment include, but are not limited to, oligonucleotide and polymerase molecules. However, the first component may be some other component such as another nucleic acid, salt, protein or enzyme.

Modulators which are detrimental to the nucleic acid replication system include, in general, molecules which compete with active components of the replications system and thus decrease its activity with respect to the nucleic acid comprised by the genetic element. For example, dideoxy-terminated primers, or inactive polymerase molecules, may be used.

In a preferred embodiment, “dummy” genes, which mimic the nucleic acid component of the genetic element and thus reduce the extent of replication thereof by competition are recruited to unmodified genetic elements. Dummy genes, which contain the same primer binding sites as the genes contained within the genetic elements, are advantageously recruited to unmodified genetic elements, e.g. to substrate molecules. When the genes are amplified (e.g. by PCR), the dummy genes compete for nucleotides and primers with the actual gene contained within the genetic element.

If there is an excess of unmodified substrate molecules within the genetic element, an excess of dummy genes will be recruited, and hence in a PCR regime where the dummy and real genes are competing for nucleotides and primers, the dummy genes will be preferentially amplified, whereas if there are no unmodified substrate molecules left within the genetic element, the gene comprised by the genetic element is amplified efficiently by PCR without competition from dummy genes.

For example, to select for a restriction enzyme acting on a specific sequence, beads are coated with both a gene encoding a mutant restriction enzyme, and with a large number of dummy genes containing the sequence one wishes to select for cleavage of; in this case, the dummy genes are not recruited as such, but contain the substrate. Active restriction enzymes cleave the dummy genes whereas inactive enzymes do not. Thus, genes encoding active restriction enzymes will be amplified without interference from dummy genes, whereas genes encoding inactive enzymes will be amplified in competition with an excess of dummy genes.

The invention moreover envisages the recruitrnent of components, including genes, nucleotides, enzymes, and the like, which are in some way detrimental to the amplification of the gene contained within the genetic element, thereby exerting a negative selection pressure.

Recruitment of Components of Nucleic Acid Replication Systems

Where the component is not itself capable of binding to modified genetic element, it will be linked to a binding moiety that can bind to modified genetic element. For example, where the first component is an oligonucleotide primer, the primer may be linked to an antibody which specifically binds to modified genetic element. The linkage may be direct, as in the case of a covalent linkage between the component and an antibody, or the component may be linked to an antibody (or other moiety which binds the modified genetic element) via one or more other molecules. For example, where the component is an oligonucleotide primer, it may be linked to a biotinylated antibody via a biotin-avidin bridge.

The linkage may also be designed to allow separation of the binding moiety from the first component. For example, an oligonucleotide primer may be linked to an antibody via a disulphide bond which may be cleaved by a reducing agent immediately prior to nucleic acid amplification. As a further example, an oligonucleotide primer may be linked to an antibody via annealing to a complimentary oligonucleotide which is linked directly to the antibody; in such a case the sequences of the oligonucleotides are designed to allow the primer to anneal preferentially to the nucleic acid gene comprised by the genetic element (rather than to the oligonucleotide linked directly to the antibdody) at the temperatures required for nucleic acid amplification.

The principle of recruiting a PCR primer specifically to the wells of antigen-coated plates by binding the primer to an antibody is used in the immuno-polymerase chain reaction (immuno-PCR) and provides a very sensitive means of antigen detection (Sano et al., 1992; Schweitzer et al., 2000).

In an alternative embodiment, the desired activity of the gene or gene product within a genetic element may result in the attachment of one or more components of a nucleic acid replication system directly to the genetic element. For example, in a bimolecular reaction, one substrate might contain or be linked to a component of a nucleic acid replication system, such that reaction of the substrates results in a product molecule which contains the component of the nucleic acid replication system. The substrate which is not linked to the component of the nucleic acid replication system may be linked to the genetic element directly or via one or more molecules which bind it, or it may become linked to the genetic element; for example, it may be linked to a protected moiety which, when deprotected, binds to the genetic element.

Comnartmnentation

Once the first component of the amplification system has been attached selectively to modified genetic element, the next stage is to perform amplification of the nucleic acid molecules contained in the modified genetic element. Selective amplification is achieved by adding to the compartments the remaining components of the amplification system which in the absence of the first component do not finction to amplify the nucleic acid molecules. For example, by way of explanation, PCR requires two oligonucleotide primers, one ‘forward’ and one ‘backward’. If the first component is the forward primer and the amplification system added to the compartments contains a second primer which is a backward primer, polymerase and dNTPs but not the first component, then amplification will only occur in compartments which contain the first primer associated with the genetic element.

Clearly, it is therefore typically necessary to remove unbound first components prior to the amplification step.

The genetic elements are subsequently divided amongst separate compartments. To the compartments are added the further components required for nucleic acid amplification (but not the first component). This may be achieved by adding a suitable mix prior to, during or after formation of the compartments.

In a preferred embodiment compartmentalisation is achieved by microencapsulating the plurality of genetic elements, for example as described in WO99/02671 and WO00/40712.

The microcapsules require appropriate physical properties. First, to ensure that the genetic elements and the components of the nucleic acid replication system may not diffuse between microcapsules, the contents of each microcapsule must be isolated from the contents of the surrounding rmicrocapsules, so that there is no or little exchange of the genetic elements or the components of the nucleic acid replication system between the microcapsules over the timescale of the experiment.

Second, the method of the present invention requires that there are only a limited number of genetic elements per microcapsule. This ensures that the bound first components of the nucleic acid replication system remain associated with the gene contained within the modified genetic element and are isolated from other genetic elements, which may or may not be modified. Thus, components of the nucleic acid replication system bound specifically to modified genetic elements will not result in the amplification of other genetic elements (which may or may not be modified). The enrichment factor is greatest with on average one or fewer genetic elements per microcapsule. However, even if the theoretically optimal situation of, on average, a single nucleic acid molecule or less per microcapsule is not used, a ratio of 5, 10, 50, 100 or 1000 or more genetic elements per microcapsule may prove beneficial in sorting a large library. Subsequent rounds of sorting, including renewed encapsulation with differing genetic element distribution, will permit more stringent sorting of the genetic elements. Preferably, there is a single genetic element, or fewer, per microcapsule. Preferably each genetic element contains only one gene or multiple copies of the same gene.

Third, the formation and the composition of the microcapsules must allow the nucleic acid replication system to function.

Consequently, any microencapsulation system used must fulfil these three requirements. The appropriate system(s) may vary depending on the precise nature of the requirements in each application of the invention, as will be apparent to the skilled person.

A wide variety of microencapsulation procedures are available (Benita, 1996)and may be used to create the microcapsules used in accordance with the present invention. Indeed, more than 200 microencapsulation methods have been identified in the literature (Finch, 1993).

These include membrane enveloped aqueous vesicles such as lipid vesicles (liposomes) (ew, 1990)and non-ionic surfactant vesicles (van Hal et al., 1996). These are closed-membranous capsules of single or multiple bilayers of non-covalently assembled molecules, with each bilayer separated from its neighbour by an aqueous compartment In the case of liposomes the membrane is composed of lipid molecules; these are usually phospholipids but sterols such as cholesterol may also be incorporated into the membranes (New, 1990).

Microcapsules can also be generated by interfacial polymerisation and interfacial complexation (Whateley, 1996). Microcapsules of this sort can have rigid, nonpermeable membranes, or semipermeable membranes. Semipermeable microcapsules bordered by cellulose nitrate membranes, polyamide membranes and lipid-polyamide membranes can all support biochemical reactions. Alginate/polylysine microcapsules (Lim & Sun, 1980), which can be formed under very mild conditions, have also proven to be very biocompatible, providing, for example, an effective method of encapsulating living cells and tissues.

Non-membranous microencapsulation systems based on phase partitioning of an aqueous environment in a colloidal system, such as an emulsion, may also be used.

Preferably, the microcapsules of the present invention are formed from emulsions; heterogeneous systems of two immiscible liquid phases with one of the phases dispersed in the other as droplets of microscopic or colloidal size (Becher, 1957; Lissant, 1974; Sherman, 1968).

Emulsions may be produced from any suitable combination of immiscible liquids. Preferably the emulsion used in the present invention has water (containing the biochemical components of the nucleic acid replication system) as the phase present in the form of finely divided droplets (the disperse, internal or discontinuous phase) and a hydrophobic, immiscible liquid (an ‘oil’) as the matrix in which these droplets are suspended (the nondisperse, continuous or external phase). Such emulsions are termed ‘water-in-oil’ (W/O). This has the advantage that the entire aqueous phase containing the biochemical components is compartmentalised in discreet droplets (the internal phase). The external phase, being a hydrophobic oil, generally contains none of the biochemical components and hence is inert.

The emulsion may be stabilised by addition of one or more surface-active agents (surfactants). These surfactants are termed emulsifying agents and act at the water/oil interface to prevent (or at least delay) separation of the phases. Many oils and many emulsifiers can be used for the generation of water-in-oil emulsions; a recent compilation listed over 16,000 surfactants, many of which are used as emulsifying agents (Ash & Ash, 1993). Suitable oils include light white mineral oil and non-ionic surfactants such as sorbitan monooleate (Span™80; ICI), polyoxyethylenesorbitan monooleate (Tween™80; ICI) and octyphenoxyethoxyethanol (Triton X-100).

The use of anionic surfactants may also be beneficial. Suitable surfactants include sodium cholate and sodium taurocholate. Particularly preferred is sodium deoxycholate, preferably at a concentration of 0.5% w/v, or below.

The addition of other molecules may also beneficial, for example proteins such as BSA. For example the efficiency of PCR was observed to increase with increasing BSA concentration, up to 10 mg/ml (see examples).

Creation of an emulsion generally requires the application of mechanical energy to force the phases together. There are a variety of ways of doing this which utilise a variety of mechanical devices, including stirrers (such as magnetic stir-bars, propeller and turbine stirrers, paddle devices and whisks), homogenisers (including rotor-stator homogenisers, high-pressure valve homogenisers and jet homogenisers), colloid mills, ultrasound and ‘membrane emulsification’ devices (Becher, 1957).

Aqueous microcapsules formed in water-in-oil emulsions are generally stable with little if any exchange of nucleic acid molecules or polypeptides between microcapsules. Additionally, it has been demonstrated that several biochemical reactions, including DNA replication, proceed in emulsion microcapsules (see WO99/02671, WO00/40712 and (Ghadessy et al., 2001; Tawfik & Griffiths, 1998)). Moreover, complicated biochemical processes, notably gene transcription and translation are also active in emulsion microcapsules (see WO99/02671, WO00/40712 and (Tawfik & Griffiths, 1998)). The technology exists to create emulsions with volumes all the way up to industrial scales of thousands of litres (Becher, 1957; Lissant, 1974; Sherman, 1968).

The preferred microcapsule size will vary depending upon the precise requirements of any individual nucleic acid replication process that is to be performed according to the present invention.

The processes of nucleic acid replication must occur within each individual microcapsule provided by the present invention.

Because of the requirement for only a limited number of DNA molecules to be present in each microcapsule, the microcapsule size will determine the size of repertoire that can be selected. The larger the microcapsule size, the larger is the volume that will be required to encapsulate a given genetic element library, since the ultimately limiting factor will be the size of the microcapsule and thus the number of microcapsules possible per unit volume. Preferably the mean volwne of the microcapsules is less than 5.2×10⁻¹³ m³, (corresponding to a spherical microcapsule of diameter less than 100 μm), more preferably less than 5.2×10⁻¹⁶ m³, (corresponding to a spherical microcapsule of diameter less than 10 μm). With 100 μm diameter microcapsules 10¹⁰ compartments (i.e. sufficient for selection of a 10¹⁰ gene library would be a total encapsulated volume of 5.2 litres. With 10 μm diameter microcapsules 10¹⁰ compartments (i.e. sufficient for selection of a 10¹⁰ gene library would be a total encapsulated volume of 5.2 ml.

The effective DNA or RNA concentration in the microcapsules may be artificially increased by various methods that will be well-known to those versed in the art. These include, for example, the addition of volume excluding chemicals such as polyethylene glycols (PEG).

It may also be necessary for the emulsion to be thermostable, preferably thermostable up to and beyond the temperatures used in thermal amplification procedures (i.e. about 95-100° C.), particularly if the nucleic acid replication system to be used requires the use of high temperatures. An example of such a thermostable emulsion is given in the Examples (a water-in oil emulsion comprising mineral oil, Span-80, Tween-80 and Triton X-100).

The microcapsule size must be sufficiently large to accommodate all of the required components of the biochemical reactions that are needed to occur within the microcapsule.

Depending on the complexity and size of the plurality of nucleic acid molecules to be screened, it may be beneficial to set up the encapsulation procedure such that one or less than one nucleic acid molecule is encapsulated per compartment, such as a microcapsule. This will provide the greatest power of resolution. Where the library is larger and/or more complex, however, this may be impracticable; it may be preferable to compartmentalise/encapsulate several nucleic acid molecules together and rely on repeated application of the method of the invention to achieve sorting of the desired binding activity. A combination of compartmentalisation/ encapsulation procedures may be used to obtain the desired enrichment.

The components for nucleic acid replication are selected for the requirements of a specific system from the following; a suitable buffer, a nucleic acid replication system containing all the necessary ingredients, enzymes and cofactors, nucleic acid polymerases, nucleotides, nucleic acids (natural or synthetic), oligonucleotides, and nucleic acid-modifying enzymes, but not the first component recruited to the modified genetic elements.

A suitable buffer will be one in which all of the desired components of the biological system are active and will therefore depend upon the requirements of each specific reaction system. Buffers suitable for biological and/or chemical reactions are known in the art and recipes provided in various laboratory texts, such as (Sambrook et al., 1989).

Once the genetic elements and the necessary components of the nucleic acid replication system have been divided amongst separate compartments, it may be necessary to increase (or decrease) the temperature of the compartments such that the nucleic acid replication system is able to replicate the genes within the genetic elements. For example, if the nucleic acid replication system used is the Polymerase Chain Reaction, it is necessary to cycle the temperature of the compartments in order to facilitate melting of duplex DNA, primer annealing and extension. As a further example, if the nucleic acid replication system used is the Self-Sustained Sequence Replication (3SR) system, it is preferable to maintain the temperature of the compartments at or near 37° C.

Once the nucleic acid replication step is complete, the nucleic acids so generated are pooled. For example, where the compartments consist of aqueous droplets in a water-in-oil emulsion, this is achieved by breaking the emulsion with a suitable reagent, such as water-saturated ether, retaining the aqueous phase.

In a highly preferred embodiment, the nucleic acid replication system used is the Polymerase Chain Reaction system, using one primer as the first component recruited to modified genetic elements. For example, in a case in which a gene product-catalysed reaction transforms substrate into product in such a way that the product becomes (or remains) associated with the genetic element, the primer component may be recruited to modified genetic elements by means of an anti-product antibody-primer conjugate (in which the primer and the antibody are physically linked). The primer may be covalently linked to the antibody (or any other ligand-specific for the product) or associated non-covalently, perhaps via on or more other components (for example, via a second antibody which recognises the first, or via another interaction such as that between biotin and avidin or streptavidin, or between complimentary sequences within the primer and an oligonucleotide linked to the antibody). In this example, nucleic acid replication of a gene within a genetic element isolated in a compartment will result in the amplification of the genes by an amount proportional to the amount of product molecule associated with the genetic element.

It may be desirable that the first component becomes separated from the binding moiety immediately before, or during, nucleic acid replication. For example, a primer may be linked to an antibody via a disulphide bond, which may be cleaved by a reducing agent immediately prior to nucleic acid amplification, or via a crosslinking agent such as disuccinimidyl tartarate which contains periodate-cleavable cis-diols. As a further example, an oligonucleotide primer may be linked to an antibody via annealing to a complimentary oligonucleotide which is linked directly to the antibody. The sequences of the oligonucleotides are designed to allow the primer to anneal preferentially to the nucleic acid gene comprised by the genetic element (rather than to the oligonucleotide linked directly to the antibody) at the temperatures required for nucleic acid amplification; this may be arranged by designing the oligonucleotide which is linked to the antibody to contain a shorter region of complementarity with the primer than the nucleic acid gene, with which the primer is complementary throughout its whole length.

In a further highly preferred embodiment, genetic elements are divided amongst separate compartments of a thermostable emulsion, with all the components of the PCR nucleic acid replication system except the primer molecule which is recruited to modified genetic elements. Thus, amplification of the gene resulting in duplex DNA copies of the gene is not able to proceed except in those compartments containing a modified genetic element to which the first component (the primer) has been recruited. In those compartments containing a modified genetic element, the gene contained within the genetic element will be amplified by an amount which is proportional to the amount of modification.

The genes obtained by this procedure may be used in a variety of ways, including, but not limited to, the following.

The genes may be further amplified by any technique, cloned by any technique, characterised by sequencing, expression or any other technique, or subjected to further rounds of selection by any technique. They may also be mutated by any technique, including, but not limited to the following. Random mutagenesis by a variety of in vivo techniques, including; using ‘mutator strains’, of bacteria such as E. coli mutD5 (Low et al., 1996); and using the antibody hypermutation system of B-lymphocytes (Yelamos et al., 1995). Random mutations can also be introduced both in vivo and in vitro by chemical mutagens, and ionising or UV irradiation (Friedberg et al., 1995), or incorporation of mutagenic base analogues (Zaccolo et al., 1996). ‘Random’ mutations can also be introduced into genes in vitro during polymerisation for example by using error-prone polymerases (Leung et al., 1989). Further diversification can be introduced by using homologous recombination either in vivo (Kowalczykowski et al., 1994) or in vitro (Stemmer, 1994a; Stemmer, 1994b; Zhao et al., 1998) or non-homologous recombination (Ostermeier et al., 1999). Such mutation may be introduced before, after, or during further amplification of the genes.

According to a further aspect of the present invention there is provided a method of in vitro evolution comprising the steps of:
(a) selecting one or more nucleic acids from a library of nucleic acids according to the present invention;
(b) mutating and/or recombining the selected nucleic acid(s) in order to generate a further library of nucleic acids, optionally encoding a repertoire of gene products;
(c) iteratively repeating steps (a) and (b) in order to obtain a gene or gene product with enhanced activity.
   Uses

The selective amplification method of the present invention may be used in a general sense to achieve quantitative amplification of a nucleic acid molecule encoding a gene product. Accordingly, in one aspect the present invention provides a method for amplifying a nucleic acid sequence encoding a gene product which method comprises
(i) providing a genetic element which contains a nucleic acid molecule which optionally encodes a gene product;
(ii) optionally expressing the nucleic acid molecule to produce the gene product and allowing the gene or gene product to modify the genetic element;
(iii) selectively attaching to modified genetic element at least a first component required for amplification of the nucleic acid molecule; and
(iv) providing further components for nucleic acid amplification, such that the nucleic acid molecule will only be amplified if it is linked to modified genetic element to which said first component has been attached;
(v) dividing the genetic elements into a number of separate compartments; and
(vi) allowing nucleic acid amplification to occur.

However, the present invention is most useful for selecting gene products having desired properties from a plurality of nucleic acid molecules, optionally encoding gene products. In particular, the methods of the present invention may be used in in vitro directed evolution techniques.

For example the methods of the present invention may be used to select nucleic acids which themselves have a desired activity, or which encode a gene product having a desired activity (which may be a novel activity) by providing a binding moiety linked to the first component of the amplification system which selectively recognises the desired modified genetic element. When a library of nucleic acid molecules is subjected to the selective amplification method of the present invention, only those nucleic acid molecules which effect the desired modification or whose encoded gene products effect the desired modification will be amplified. Furthermore, those nucleic acid molecules, or whose encoded gene products, effect the desired modification most efficiently, will be amplified the most. Thus, desired gene products can be isolated from the selectively amplified gene population.

In another embodiment, the selective gene amplification system may be used in conjunction with directed in vitro evolution of a characterised gene product to improve or modify its properties. In this case, the plurality of nucleic acid molecules may have been subjected to site-directed mutagenesis to target specific regions of the gene product, or to completely random mutagenesis over the entire coding sequence. The plurality of nucleic acid molecules may also have been subjected to homologous or non-homologous recombination. The library encoding the gene product and variants (mutants) thereof may then be tested using the selective amplification system of the present invention. Variants that are amplified to a different extent than the original umnutated gene product may then be selected. For example, variants that are more active than the original unmutated gene product may be selected by virtue of their increased amplification.

Multiple rounds of selection/amplification may be used to obtain enrichment of a desired gene and the gene(s) may be subjected to further rounds of mutation and/or recombination between rounds of selection/amplification. Thus relatively modest increases in activity may lead rapidly to large amounts of amplified gene product over a number of rounds.

The present invention also provides kits for carrying out the methods of the present invention. Kits of the invention typically comprise: (i) a plurality of genetic element optionally encoding a plurality of gene products, each genetic element comprising a coding sequence operably linked to a regulatory sequence which is capable of directing expression of a gene product encoded by the sequence, wherein the desired activity of the gene or gene products results, directly or indirectly, in the modification of the genetic elements which contained/encoded them; (ii) a first component required for amplification of the nucleic acid constructs which is capable of selectively binding to genetic elements modified by the action of the gene or gene products but not to unmodified genetic elements.

Typically, the first component is a nucleic acid primer capable of priming polynucleotide polymerisation from the nucleic acid constructs. The plurality of nucleic acid constructs are typically comprised as described above for pluralities of nucleic acid molecules. For example, the plurality of nucleic acid constructs may encode a gene product and variants thereof to be tested for a given activity. In a preferred embodiment, the genetic element comprises the gene attached to a solid phase, such as a microsphere, and optionally a variety of other molecules.

Kits may also comprise suitable buffers, packaging and instructions for using the kit.

The present invention will now be described by way of the following examples, which are illustrative only and non-limiting.

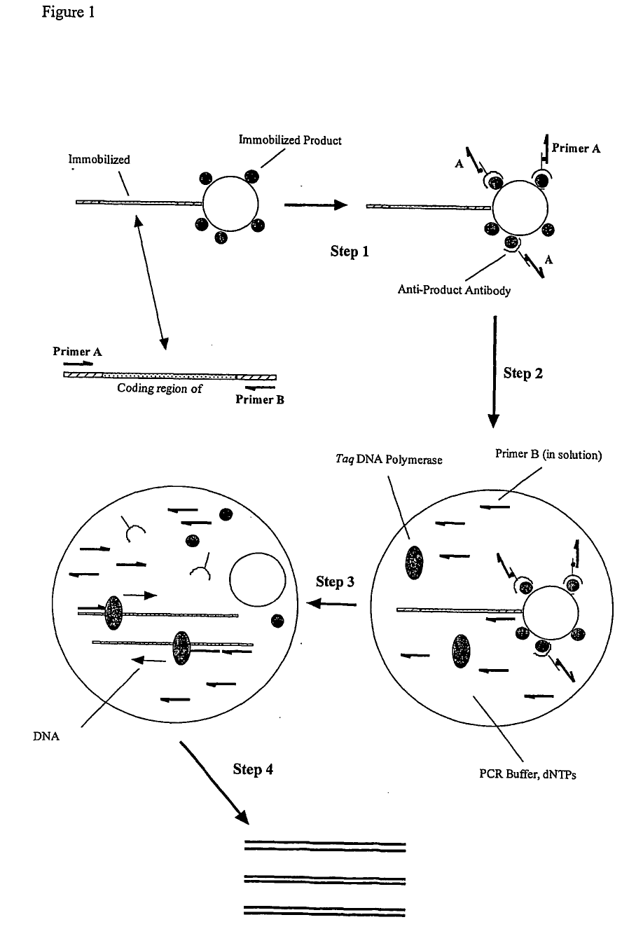

In a general example of the invention, an active mutant enzyme within the compartment of an emulsion has catalysed the transformation of a certain number of substrate molecules into product molecules, which are immobilised on the surface of a microsphere along with the gene encoding the mutant, as depicted in FIG. 1. Anti-product antibodies, each linked to several copies of primer A, which primes within the sequence of the template gene (in the conserved sequence region outside the gene), are allowed to bind to the product molecules displayed on the surface of each microsphere (after the emulsion is broken) (Step 1). Thus, a certain number of primers, proportional to the number of product molecules, are recruited to each microsphere. The microspheres are then mixed with a PCR reaction mixture containing only primer B and dispersed amongst the droplets of an emulsion which is thermostable up to at least a temperature of 94° C. (Step 2). Together, primers A and B will allow the amplification of the gene. The temperature of the emulsion is then cycled in order to allow the PCR reaction to occur within the compartments of the emulsion, which are stable and will not coalesce. An asymmetric PCR will occur in each compartment (Step 3); the number of copies of the gene made will be limited by the number of copies of primer A present—in other words, the maximum number of copies of the gene made will be a fixed multiple of the number of product molecules formed by the protein it encodes. After cycling, the emulsion is broken (Step 4) and the genes further amplified in a nesting PCR process if necessary.

For example, in a hypothetical situation there may be 100 “+ve” genes for every one “+ve” gene (where the −ve gene encodes a catalytically inactive mutant, and the +ve gene encodes a catalytically active mutant). The enzyme encoded by the +ve gene catalyses the formation of 100 molecules of product, whereas the protein encoded by the −ve gene does not increase the rate of product formation over background, which gives perhaps 5 molecules of product. After the PCR procedure outlined above, each +ve gene will be amplified 100-fold (if each anti-product antibody carries a single copy of primer A) and each −ve gene will be amplified 5-fold. Thus the new ratio of −ve to +ve genes will be 5:1 rather than 100:1, an enrichment of 20-fold, which corresponds to the ratio of the catalysed turnover to the uncatalysed turnover.

This scheme links the fitness (i.e. the reproductive success, or number of progeny) of the gene directly to the turnover of the enzyme it encodes, thus focusing selection directly on the gene in a manner analogous to the way in which natural selection acts on whole organisms.

In the model system investigated here, biotinylated primers are immobilised on the surface of a streptavidin-coated microsphere which carries the “positive” gene (representing a gene which encodes an active enzyme). Microspheres carrying the “negative” gene (representing a gene which does not encode active enzyme) are mixed with these “positive” microspheres such that there is a 100-fold excess of the negatives over the positives.

Initial experiments show that a 100-fold enrichment of the desired gene may be obtained using between 5,000 and 10,000 primers immobilised per “positive” microsphere. Further optimisation is to be carried out, directed towards increasing the efficiency of the PCR within the compartments of the emulsion. In addition, it might be possible to introduce many more primers per molecule of immobilised product, for example by immobilising the anti-product antibody itself on a very small microsphere (less than 0.1 μm diameter) along with a large number of primer molecules.

### Materials and Methods

Primers

Primers annealing upstream of T7 promoter, pIVEX vectors

(in order of decreasing distance upstream from T7 promoter):

| | |
|---|---|
| lmb2-1 | 5′ biotin-CAGGCGCCATTCGCCATT 3′ |
| lmb2-5 | 5′ biotin-CCAGCTGGCGAAAGGGGG 3′ |
| lmb2-7 | 5′ biotin-AAGTTGGGTAACGCCAGG 3′ |
| lmb2-7sul | 5′ thiol-AAGTTGGGTAACGCCAGG 3′ |

Primers (pIVB Series) annealing downstream of expressed gene, pIVEX vectors

(in order of increasing distance downstream of gene):

| | |
|---|---|
| pIVB9 | 5′ TATCCGGATATAGTTCC |
| pIVB8 | 5′ CACACCCGTCCTGTGGA |
| pIVB7 | 5′ GTCGCCATGATCGCGTA 3′ |
| pIVB5 | 5′ CCTGCTCGCTTCGCTAC 3′ |
| pIVB1 | 5′ AGAAATTGCATCAACGC 3′ |

Microspheres—Properties and Handling

The microspheres used herein are of two kinds: magnetic microspheres (diameter 2.8 μm, Dynal) and non-magnetic, polystyrene microspheres (diameter 0.96 μm, Bangs Laboratories). Both types are in the form of colloidal suspensions, and the microspheres are easily dispersed throughout the aqueous phase by gentle pipetting. The magnetic microspheres are easily separated from the aqueous phase by means of a magnet, whereas the polystyrene microspheres must be centrifuged gently, thus forming a pellet, before removal of the aqueous phase (supernatant).

To determine the concentration of a sample of microspheres, the OD₆₀₀ of a suitable dilution of the microspheres in a volume of 1 ml PBS is determined using a Pharmacia Biotech Ultrospec 3000 spectrophotometer. Comparison against a standard curve allows the concentration to be determined.

Washing Microspheres

To wash polystyrene microspheres, the suspension is centrifuged at 5,000 rpm in an MSE Micro Centaur variable-speed microcentrifuge, for 2 minutes. The supernatant is removed with a pipette and the pellet resuspended in the wash buffer.

Some buffers are problematic in that microspheres cannot be spun down particularly effectively in them. Such buffers include those with high concentrations of salt, such as Washing and Binding buffer (5 mM Tris, pH 7.4; 0.5 mM EDTA; 1M NaCl). In these cases, after a brief incubation with the microspheres, the suspension may be diluted fivefold in water. In most cases the microspheres may then be handled normally.

Immobilising biotinylated DNA & proteins on Streptavidin-coated microspheres

Biotinylated DNA is incubated at the appropriate concentration with microspheres in TNT buffer (0.1 M Tris, pH 7.5; 0.15 M NaCl; 0.05% Tween-20), for one hour at room temperature. The efficiency with which streptavidin-coated microspheres bind DNA is approximately 50%; thus, for a final concentration of 1 gene per microsphere, the microspheres are incubated with a twofold molar excess of biotinylated genes.

PCR

PCR reaction mixtures contained each primer at 500 nM; dNTPs at 250 μM each; 1xSuperTaq buffer; 2.5 units SuperTaq (HT Biotechnology). Small-scale plasmid preparations were generally diluted 100-fold or 500-fold in water and used as a 20x template concentrate.

Each PCR cycle generally consisted of the following steps: 94° C. for 1 minute; 55° C. for 1 minute; 72° C. for 2 minutes. Reactions were normally cycled 25 times, followed by an additional 5 minute extension phase (at 72° C.).

The concentrations of PCR products were determined by measuring OD₂₆₀ using a Pharmacia Biotech Ultrospec 3000 spectrophotometer.

Emulsions—Span/Tween

Prepare an oil mix by mixing 95 ml Mineral Oil, 4.5 ml Span-80 Fluka) and 0.5 ml Tween-80, stirring or shaking until clear. Dispense 1 ml to a 2 ml Biofreeze tube (Corning) containing a small magnetic stir-bar and chill on ice for at least 15 minutes prior to making the emulsion. To make the emulsion, pipette 5×10 μl aliquots of the aqueous phase into the oil whilst stirring at 1400 rpm, without waiting between each aliquot. After the final aliquot has been added to the oil mix, continue stirring for 1 minute. Do not stir for longer. Once the emulsion is formed the mixture should become opaque.

Emulsions—SpanlTriton

Prepare an oil mix by mixing 38 g of Mineral Oil, 1.8 g Span-80 (Fluka) and 0.2 g Triton X-100 (Fisher), stirring or shaking until clear. Dispense 500 μl of the oil mix to a 2 ml Biofreeze tube (Corning) containing a small magnetic stir-bar and chill on ice for at least 15 minutes prior to making the emulsion. To make the emulsion, pipette 50 μl of the aqueous phase directly in the emulsion (i.e. not in aliquots), whilst stirring at 1000 rpm. Continue stirring for 5 minutes. Do not stir for longer.

Breaking Emulsions (Span/Tween & Span/Triton)

Transfer the emulsion into a suitable tube. Spin down at full speed in a microcentrifuge for 1 minute; remove as much of the clearer fraction as possible. Resuspend the remainder (which should still be opaque) in 200 μl wash buffer (TNT). Add 1 ml hexane. Shake vigorously. Spin in a microcentrifuige at full speed for about 10 seconds. Remove the clearer upper layer. Add 1 ml hexane, repeat. Repeat this process until the lower layer is no longer opaque. If there are a sufficient number of microspheres present, a pellet may be visible. Finally, remove residual hexane by spinning the tube in a Speedvac.

Emulsions—Thermostable

Prepare an oil mix by mixing 95 ml Mineral Oil, 4.5 ml Span-80 (Fluka), 0.4 ml Tween-80 and 0.05 ml Triton X-100 (Fisher), stirring or shaking until clear. Dispense 400 μl of the oil mix to a 2 ml Biofreeze tube (Corning) containing a small magnetic stir-bar. Do not chill; leave at room temperature. To make the emulsion, add 200 μl of aqueous phase dropwise over 1.5 minutes, stirring at 1000 rpm. Continue stirring for 5 minutes thereafter. Do not stir for longer.

Breaking Emulsions (Thermostable)

Add two volumes (1.2 ml) of ether to the oil mix in a suitable tube, vortex and spin in a microcentriflge for 1 minute at fuill speed. Remove the upper phase; retain the lower phase.

PCR In Emulsion Compartments

The OPD-HA gene used as a template was amplified from the vector pIVBX-OPD-HA using the primers lmb2-1 and pIVB1.

A PCR reaction mixture is prepared in a total volume of 250 μl. 200 μl is used to form a thermostable water-in-oil emulsion as described in section above. Once the emulsion is formed, it is aliquoted into the wells of a microtitre plate (approximately 90 μl in each well) and each aliquot is overlaid with mineral oil. It is important to overlay with oil, despite the fact that the emulsion is largely composed of mineral oil; without an additional top layer, the topmost part of the emulsion breaks during thermocycling.

The emulsion is cycled, each cycle consisting of the following steps: 94° C. for 1 minute; 55° C. for 1 minute; 72° C. for 4 minutes. Reactions are cycled 25 times followed by an additional 5 minute extension phase (at 72° C.). The aliquots are removed from the plate, pooled and broken as described above.

### EXAMPLE 1

### Asymmetric PCR In Solution Using a Biotinylated Primer and Template Immobilised to a Microsphere.

Asymmetric PCR

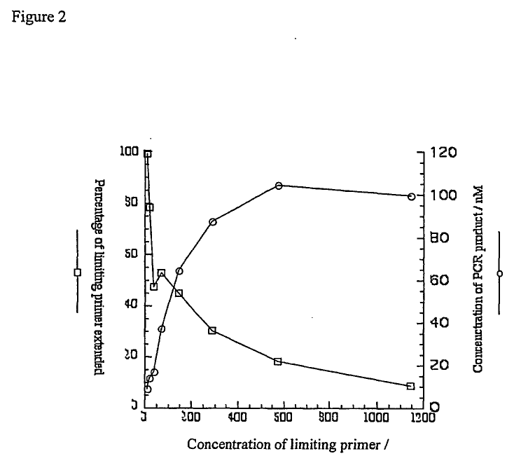

To investigate the feasibility of successful asymmetric PCR within the compartments of an emulsion, a simple PCR experiment is performed (in solution), in which the concentration of one primer was titrated down while the concentration of the second primer was kept fixed. The percentage of the limiting primer which is extended is estimated by gel electrophoresis which showed that at lower concentrations of the limiting primer, a higher proportion of that primer is extended (FIG. 2). Thus, such an asymmetric PCR will still result in primer extension at the low primer concentrations envisaged in the emulsion-PCR selection scheme.

Model System—Primers Recruited via Avidin-Biotin

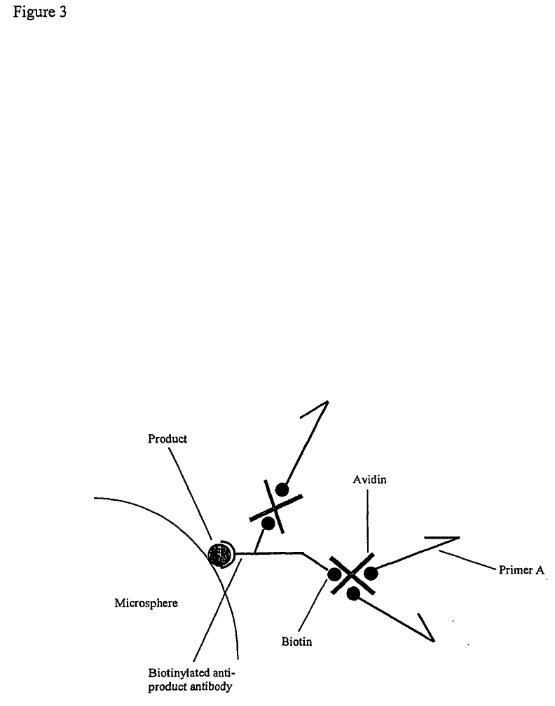

We have found that streptavidin immobilised on microspheres releases biotinylated DNA upon heating at 90° C. Thus, one approach to the system described above is to bind primers to a biotinylated anti-product antibody via an avidin bridge as shown in FIG. 3.

During the denaturation cycle of the PCR reaction, the primers immobilised in this way are likely to dissociate from the antibody; even if they are unable to do so, the antibody will dissociate from the product and become denatured, thereby allowing the primer to associate with the gene.

As a model system, biotinylated primers are immobilised directly onto a streptavidin-coated microsphere, along with one copy of a gene.

Biotinylated Primers Immobilised On Microspheres Are Extended In a Solution PCR Reaction

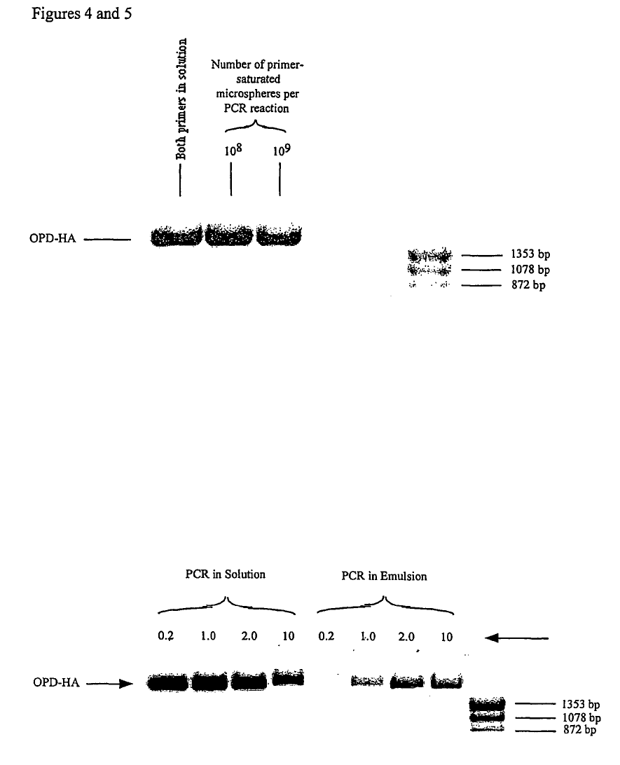

Primer A (lmb2-1.bio) is immobilised on streptavidin-coated microspheres at saturating concentration. The microspheres are washed with a high salt buffer to ensure removal of unbound primer. PCR reactions are prepared, one containing 10⁹ microspheres, one containing 10⁸ nricrospheres and one containing the lmb2-1.bio primer in solution (with no microspheres present). All the reactions are in 50 μl volumes. The gene OPD-HA, amplified from the vector pIVEX-OPD-HA with primer A (lmb2-1.bio) and primer B (pIV-B1), is introduced as the template, at a concentration of 0.02 nM. The reactions are cycled with a standard PCR protocol. Products are visualised by gel electrophoresis (FIG. 4).

This experiment conmms that immobilised, biotinylated primers are capable of extension in a PCR reaction, and that the primers are released during the reaction (microspheres do not migrate in an agarose gel).

### EXAMPLE 2

### PCR In Emulsion Compartments

The OPD-HA gene is used as the template in an emulsion reaction. PCR reactions, with both primers A and B in solution are set up, emulsified and cycled using a protocol modified to include a slightly longer extension phase (the emulsion may take longer to rise to the desired temperature), with the inclusion of varying concentrations of BSA. The products are visualised by gel electrophoresis (FIG. 5). The efficiency of the PCR increases with increasing BSA concentration, up to 10 mg/ml (the maximum concentration used).

Model Selection

The OPD-HA gene is chosen as the +ve gene and the DHFR gene as the −ve. Four populations of microspheres are prepared, as follows:
1. Microspheres carrying 1 DHFR gene each;
2. Microspheres carrying 1 OPD-HA gene each;
3. Microspheres carrying 1 OPD-HA gene each, incubated subsequently with a 1000-fold molar excess of biotinylated primer A (lmb2-1.bio), likely to yield between 500 and 1000 primers per microsphere.
4. As (3) above, but incubated with a 10,000-fold molar excess, likely to yield between 5000 and 10,000 primers per microsphere.

Thus, the microspheres coated with both the OPD-HA gene and the primers represent cases in which anti-product antibodies carrying primers have bound to microspheres.

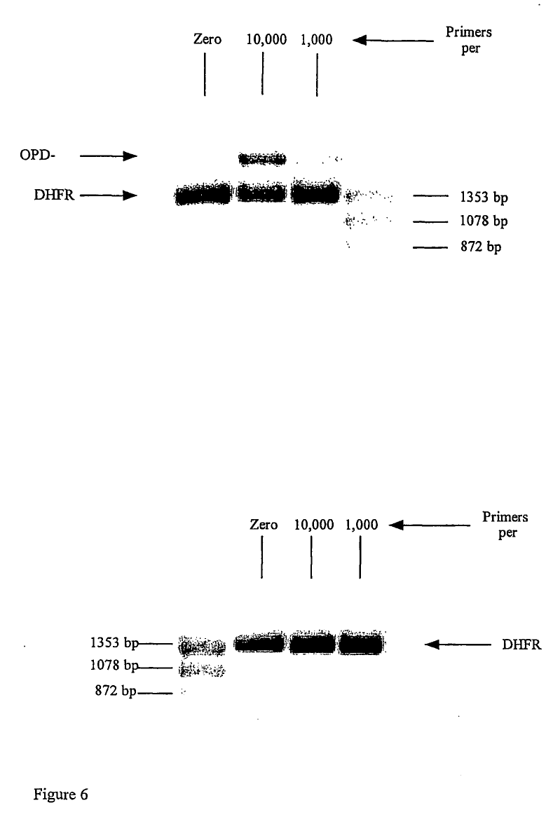

Three mixed populations are prepared, each containing a 1:100 ratio of OPD-HA-carrying microspheres to DHFR-carrying microspheres; one mixed population contains microspheres with the OPD-HA gene alone, whereas the other two contain microspheres with both the OPD-HA gene and the immobilised primer A, at the two different concentrations described in (3) and (4) above. 10⁸ microspheres from each mixed population are resuspended in PCR reaction mix lacking primer A but containing primer B, and 10 mg/ml BSA. Each PCR mix is subsequently split into two aliquots; one aliquot is emulsified and cycled above whereas the other is left in bulk solution and cycled in the same way. Emulsions are then broken, and nesting PCR reactions performed using the primers lmb2-5.bio and pIV-B7, in solution, with 1 μl of the recovered emulsion-PCR reactions, or 1 μl of the bulk solution PCR reactions, as template. The products are visualised by gel electrophoresis (FIGS. 6A and 6B).

In the case of the PCR reactions carried out in solution (FIG. 6A), only the PCR product corresponding to the DHFR gene is visible, indicating no enrichment of the OPD-HA gene. However, in the case of the PCR reactions carried out in the compartnents of a thermostable emulsion (FIG. 6B), there is visible enrichment of the OPD-HA gene in each of the two reactions containing microspheres which carried the primer lmb2-1.bio in addition to the OPD-HA gene, almost 100-fold in the case of the microspheres coated with 5,000-10,000 primers. Where the OPD-HA gene is immobilised on microspheres carrying no primers, there is no enrichment.

In bulk solution, primers which have been extended by DNA polymerase are free to diffuse throughout the reaction volurne, so that primers co-immobilised with the OPD-HA gene may diffluse away to prime off the DHFR gene, whereas in a compartment of an emulsion, the primers are not free to diff-use to other compartments, so that only the co-immobilised OPD-HA gene is amplified, leading to a selective enrichment of the OPD-HA gene. The experimental results demonstrate that compartmentalisation is occurring within the thermostable emulsion. An enrichment of almost 100-fold has been achieved in the case of the microspheres coated with 5,000-10,000 primers. Further optimisation can increase the efficiency of enrichment; for example, droplets formed in the thermostable emulsion are larger than those formed in the emulsions used in previous experiments, indicating that the local primer concentration in the vicinity of a given microsphere can be increased by reducing the mean diameter of the emulsion droplets.

### EXAMPLE 3

### Quantitation Of Asymmetric PCR In Emulsions with Varying Quantities Of Limiting Primer Immobilized On Beads

Biotinylated DNA encoding the N-FLAG-OPD-HA gene is immobilized on 1 μm diameter streptavidin-coated microspheres at a concentration of I gene per rnicrosphere. Biotinylated primer lmb2-7 is then immobilized on the microspheres at various concentrations. The microspheres are washed and subsequently 10⁷ icrospheres are transferred to a 200 μl PCR reactions which are then emulsified and cycled as in the previous example. The emulsions are broken and the aqueous phase retrieved with the addition of 200 μI 25 μg/ml yeast tRNA (Sigma) to act as a carrier for the DNA.

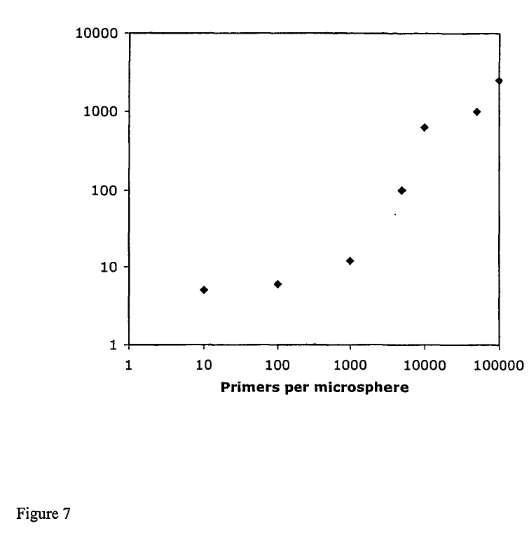

The amount of DNA amplifiable by PCR is quantitated by competitive PCR essentially as described in (Gilliland et al., 1990) with the gene DHFR-HA (amplified from the vector pIVEX-DHFR-HA with the primers lmb2-5 and pIVB5) as competitor, using the primers lmb2-7 and pIVB9. FIG. 7 shows the results.

### EXAMPLE 4

### Preparation Of Antibody-Neutravidin Conjugates for Recruitment Of Biotinylated Primers

In order to provide a means of recruiting a biotinylated primer to an antibody (in turn recruited to a product-specific antibody), a chemically crosslinked antibody-neutravidin conjugate is prepared as follows.

1. Add 50 μl iminothiolane (Sigma) at 5 mg/ml in DMF to 1 ml goat anti-rabbit IgG (minimal cross reactivity with human, mouse and rat serum proteins; Jackson) at a concentration of 1.8 mg/ml in 0.01 M phosphate, 0.25 M NaCl, pH 7.6, and incubated for 30 minutes at 37° C. with mixing. The antibody is purified on a PD-10 column (AP Biotech), eluting in PBS.

2. Dissolve 2.5 mg sulfo-SMCC (Pierce) in 50 μl DMSO and add to 750 μl neutravidin (Pierce) at 10 mg/ml in water. React for 1 hour at room temperature with mixing. Purify on a PD-10 column, eluting in PBS.

3. Mix together the neutravidin and the antibody in a 5:1 or 10:1 molar ratio (neutravidin in excess) and react for 1 hour at 37° C. followed by overnight at 4° C.

4. Purify using a FreeZyme conjugate purification kit (Pierce), as per manufacturer's instructions.

5. Stabilize the conjugate with the addition of 1 mg/ml BSA, add glycerol to 50%, aliquot, and store at −20° C.

### EXAMPLE 5

### Recruitment Of Biotinylated Primers with Antibody-Neutravidin Conjugate

In one format of the selection, primers linked to an antibody are recruited to product molecules linked to a microsphere carrying a gene encoding an active enzyme, but not to substrate molecules linked to a microsphere carrying a gene encoding an inactive enzyme. Here this process is simulated.

The enzyme phosphotriesterase catalyses the hydrolysis of EtNP-Bz-Glu-biotin, a biotinylated substrate molecule prepared essentially as described in WO00/40712. Product molecules linked to microspheres may be detected by fluorescent anti-product antibodies raised in rabbits as described in WO00/40712. Alternatively, non-fluorescent rabbit antibodies bound to product molecules may be detected by the addition of anti-rabbit IgG molecules (AffmiPure goat anti-rabbit IgG, minimal cross-reactivity with human, mouse and rat serum proteins; Jackson Inmuno Research).

To prepare the antibody-neutravidin-primer complex, 13 μl of primer lmb2-7 (at 10 μM concentration) is added to 30 μl antibody-neutravidin stock (prepared as in Example 4) and incubated for 45 minutes at room temperature. 1 μl of D-biotin (at a concentration of 500 μM) is added to block any remaining biotin-binding sites, and the mixture is incubated for a further 30 minutes at room temperature.

Microspheres are coated with the gene N-FLAG-OPD-HA (amplified with the primers lmb2-1 and pIVB1 from the vector pIVEX-N-FLAG-OPD-HA) at a concentration of one gene per microsphere. The microspheres are further incubated with approximately 3×10⁶ molecules of biotinylated substrate, biotinylated product or biotin, per microsphere. The microspheres are then washed 3 times with PBS/T (PBS containing 0.1% Tween-20) and finally resuspended in 10 times their original volume of PBS/T. A 1 in 30 dilution of rabbit serum (containing the anti-product antibody) in COVAp buffer (2M NaCl, 0.04% Tween-20, 10 mM phosphate, 0.1 mM p-nitrophenol, pH 6.5), with 1.5 mg/ml BSA, is added in an equal volume to the microspheres. After a 1 hour incubation at room temperature, with shaking, the microspheres are washed 3 times in PBS/T and resuspended in 10 times their original volume of PBS/T. The microspheres are placed in a sonicating water bath for 1 minute. 1 μl D-biotin is then added per 10⁸ microspheres and the mixtures incubated at room temperature for 10 minutes with shaking. 13 μl of the antibody-neutravidin-primer mixture is added in a total volume of 50 μl in COVAp with 1.5 mg/ml BSA, per 10⁸ microspheres. The microspheres are incubated for 1 hour at room temperature with shaking, washed three times in PBS/T, once in SuperTaq buffer (EI Biotechnology), and finally resuspended in 50 μl SuperTaq buffer per 10⁸ microspheres.

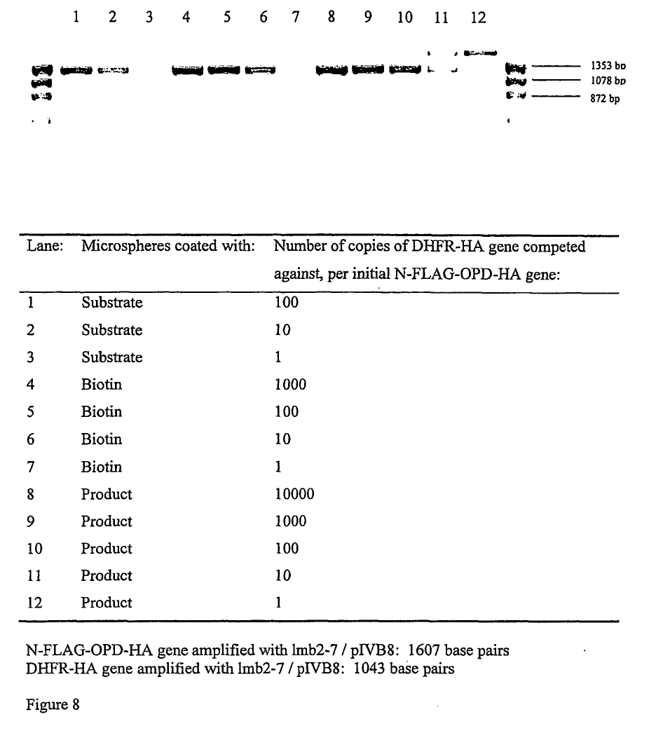

1.25×10⁷ microspheres are added to a PCR reaction mixture containing the primer pIVB8 (with no lmb2-7) in a volume of 250 μl. 200 μl of each PCR mixture is used as the aqueous phase in a thermostable emulsion (see above) and cycled 25 times. The emulsions are broken, the aqueous phases retrieved, and the degree of amplification of the N-FLAG-OPD-HA gene assessed by competitive PCR, essentially as described in (Gilliland et al., 1990) with the gene DHFR-HA (amplified from the vector pIVEX-DHFR-HA with the primers lmb2-5 and plVB5) as competitor, using the primers lmb2-7 and pIVB9. The results, shown in FIG. 8, indicate that the N-FLAG-OPD-HA genes linked to microspheres carrying biotinylated product have been amplified more than those linked to microspheres carrying biotinylated substrate or biotin. This suggests that the antibody-neutravidin-primer complex may be used to recruit primers specifically to product molecules.

### EXAMPLE 6

### Preparation Of Antibody-Oligonucleotide Conjugates

Oligonucleotides linked directly to an antibody are prepared as follows.

1. 1.5 mg sulfo-SMCC (Pierce) is added to 4.5 mg AffiniPure goat anti-mouse IgG (Jackson Immuno Research), in a volume of 2.5 ml (0.01 M phosphate, 0.25 M NaCl, pH 7.6), and incubated for 1 hour at room temperature with mixing.

2. Unreacted SMCC is removed using a PD-10 column (AP Biotech), eluting in 3.5 ml PBS.

3. Approximately 140 nmoles of thiolated oligonucleotide (lmb2-7sul) is added and the solution incubated for 2 hours at room temperature with mixing, followed by overnight at 4° C.

4. The conjugate is purified on a FreeZyme conjugate purification column (Pierce), following the manufacturer's instructions.

5. The conjugate is further purified on a PD-10 column (AP Biotech), eluting in 3.5 ml TBS.

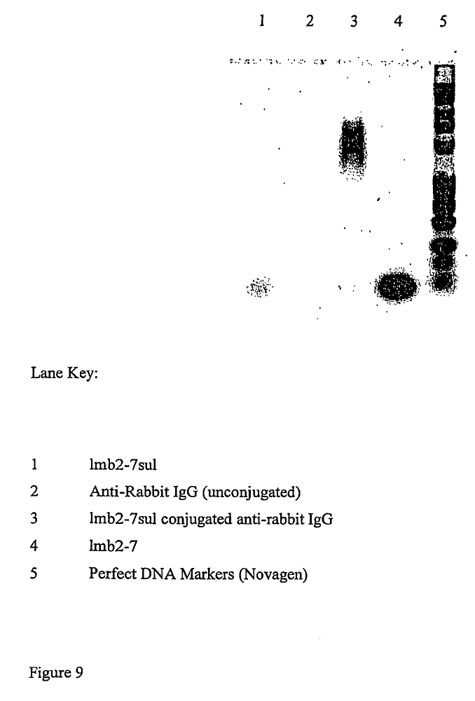

5. To visualize linkage of the oligonucleotide, the purified conjugate is run on a 1.5% agarose/TBE gel, with 0.5 μl GelStar (BMA Products) per 100 ml gel, and compared with oligonucleotides lmb2-7 and lmb2-7sul and with unconjugated antibody (FIG. 9).

### EXAMPLE 6

### Selective Amplification Of Genes

Here, a gene, associated with product molecules, is enriched from an excess of genes, associated with substrate molecules.

Microspheres are initially coated with 2500 molecules of biotinylated anti-HA antibody (Roche, 3F10), to simulate a selection in which enzymes encoded by a gene are captured on the surface of the microspheres.

Microspheres are further coated with the gene DHFR-HA (amplified from the vector pIVEX-DHFR-HA with the primers lmb2-5 and plVB5) at a concentraton of one gene per microsphere, or with the gene N-FLAG-OPD-HA at the same concentration. Microspheres coated with the DHFR-HA gene are further incubated with approximately 3×10⁶ molecules of biotinylated substrate per microsphere, whereas microspheres coated with the N-FLAG-OPD-HA gene are fither incubated with a similar quantity of biotinylated product.

The microspheres are then washed 3 times with PBS/T (PBS containing 0.1% Tween-20) and finally resuspended in 10 times their original volume of PBS/T. A 1 in 30 dilution of rabbit serum (containing the anti-product antibody) in COVAp buffer (2M NaCl, 0.04% Tween-20, 10 mM phosphate, 0.1 mM p-nitrophenol, pH 6.5), with 1.5 mg/ml BSA, is added in an equal volume to the microspheres. After a 1 hour incubation at room temperature, with shaking, the microspheres are washed 3 times in PBS/T and resuspended in 10 times their original volume of PBS/T. The microspheres are placed in a sonicating water bath for 1 minute. 1 μl D-biotin is then added per 10⁸ microspheres and the mixtures incubated at room temperature for 10 minutes with shaking. 13 μl of the antibody-neutravidin-primer mixture (see Example 5) is added in a total volume of 50 μl in COVAp with 1.5 mg/ml BSA, per 10⁸ microspheres. The microspheres are incubated for 1 hour at room temperature with shaking, washed three times in PBS/T, once in SuperTaq buffer (HT Biotechnology), and finally resuspended in 50 μl SuperTaq buffer per 10⁸ microspheres.

The microspheres are mixed in the ratios 1:10, 1:100 and 1:1000 (N-FLAG-OPD-HA gene coated microspheres : DHFR-HA gene coated microspheres).

1.25×10⁷ microspheres are added to a PCR reaction mixture containing the primer pIVB8 (with no lmb2-7) in a volume of 250 μl. 200 μl of each PCR mixture is used as the aqueous phase of a thermostable emulsion (see above) and cycled 25 times. The emulsions are broken, the aqueous phases retrieved, and an aliquot taken as the template in a futher PCR reaction using the primers lmb2-7 and plVB9. For comparison, aliquots of the microsphere mixtures are placed directly into a similar PCR reaction, without first being amplified in an emulsion.

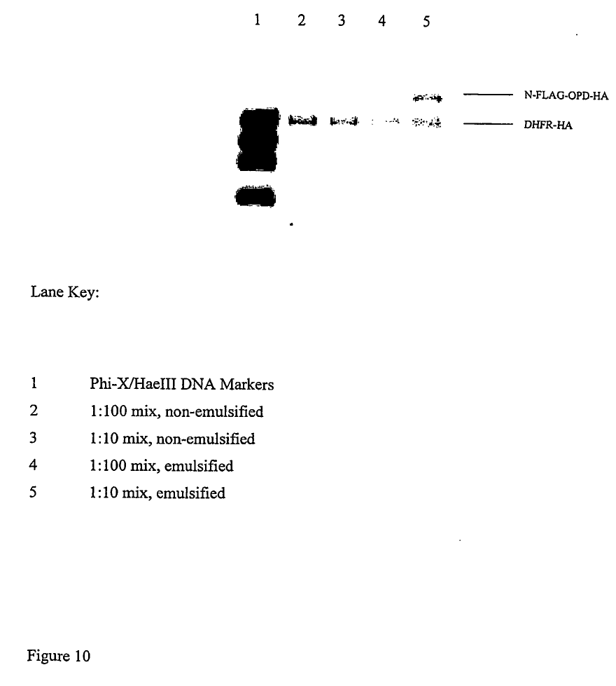

Aliquots of these PCR reactions are visualised by agarose gel electrophoresis as shown in FIG. 10. In the case of the 1:10 microsphere mixture used in an emulsified PCR reaction, the N-FLAG-OPD-HA gene has clearly been enriched compared to the DHFR-HA gene, as can be seen by comparison with the 1:10 microsphere mixture amplified directly (without compartmentation in an emulsion).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are readily apparent to those skilled in molecular biology or related fields are intended to be within the scope of the following claims.

### References

Arkin, M. R. & Wells, J. A. (1998). Probing the importance of second sphere residues in an esterolytic antibody by phage display. J Mol Biol 284(4), 1083-94.
Ash, M. & Ash, I. (1993). Handbook of industrial surfactants, Gower, Aldershot.
Atwell, S. & Wells, J. A. (1999). Selection for improved subtiligases by phage display. Proc NatlAcadSci USA 96(17), 9497-502.
Barany, F. (1991). Genetic disease detection and DNA amplification using cloned thermostable ligase. Proc NatlAcadSci USA 88(1), 189-93.
Becher, P. (1957). Emulsions: theory andpractice, Reinhold, New York.
Benita, S., Ed. (1996). Microencapsulation: methods and industrial applications. Drugs and pharmaceutical sciences. Edited by Swarbrick, J. New York: Marcel Dekker.
Boder, E. T. & Wittrup, K. D. (1997). Yeast surface display for screening combinatorial polypeptide libraries. Nat Biotechnol 15(6), 553-7.
Compton, J. (1991). Nucleic acid sequence-based amplification. Nature 350(6313), 91-2.
Demartis, S., Huber, A., Viti, F., Lozzi, L., Giovannoni, L., Neri, P., Winter, G. & Neri, D. (1999). A strategy for the isolation of catalytic activities from repertoires of enzymes displayed on phage. Journal of Molecular Biology 286(2), 617-633.
Fastrez, J. (1997). In vivo versus in vitro screening or selection for catalytic activity in enzymes and abzymes. Mol Biotechnol 7(1), 37-55.
Finch, C. A. (1993). Encapsulation and controlled release. Spec. Publ.-R. Soc. Chern. 138, 35.
Friedberg, E. C., Walker, G. C. & Siede, W. (1995). DNA repair and mutagenesis, ASM Press, Washington D.C.
Georgiou, G., Stathopoulos, C., Daugherty, P. S., Nayak, A. R., Iverson, B. L. & Curtiss, R., 3rd. (1997). Display of heterologous proteins on the surface of microorganisms: from the screening of combinatorial libraries to live recombinant vaccines. Nat Biotechnol 15(1), 29-34.
Ghadessy, F. J., Ong, J. L. & Holliger, P. (2001). Directed evolution of polymerase function by compartmentalized self-replication. Proc NatlAcad Sci USA 98(8), 4552-7.
Gilliland, G., Perrin, S., Blanchard, K. & Bunn, H.F. (1990). Analysis of cytokine MRNA and DNA: Detection and quantitation by competitive polymerase chain reaction. Proc Natl Acad Sci USA 87(7), 2725-9.
Guatelli, J. C., whitfield, K. M., Kwoh, D. Y., Barringer, K. J., Richman, D. D. & Gingeras, T. R. (1990). Isothermal, in vitro amplification of nucleic acids by a multienzyme reaction modeled after retroviral replication. Proc Natl Acad Sci USA 87(5), 1874-8.
Ho, S. N., Hunt, H. D., Horton, R. M., Pullen, J. K. & Pease, L. RF (1989). Site-directed mutagenesis by overlap extension using the polymerase chain reaction. Gene 77(1), 51-9.
Horton, R. M., Hunt, H. D., Ho, S. N., Pullen, J. K. & Pease, L. R. (1989). Engineering hybrid genes without the use of restriction enzymes: gene splicing by overlap extension. Gene 77(1), 61-8.
Innis, M. A., Gelfand, D. H., Sninsky, J. J. & White, T. J., Eds. (1990). PCR Protocols: A guide to methods and applications. New York: Academic Press.
Janda, K. D., Lo, L. C., Lo, C. H. L., Sim, M. M., Wang, R., Wong, C. H. & Lemer, R. A. (1997). Chemical selection for catalysis in combinatorial antibody libraries. Science 275(5302), 945-8.
Jermutus, L., Ryabova, L. A. & Pluckthun, A. (1998). Recent advances in producing and selecting functional proteins by using cell-free translation. Curr Opin Biotechnol 9(5), 534-48.
Jestin, J. L., Kristensen, P. & Winter, G. (1999). A method for the selection of catalytic activity using phage display and proximity coupling. Angew Chem Int Ed 38(8), 1124-1127.
Kowalczykowski, S. C., Dixon, D. A., Eggleston, A. K., Lauder, S. D. & Rehrauer, W. M. (1994). Biochemistry of homologous recombination in Escherichia coli. Microbiol Rev 58(3), 401-65.
Lancet, D., Sadovsky, E. & Seidemaum, E. (1993). Probability model for molecular recognition in biological receptor repertoires: significance to the olfactory system. Proc Natl Acad Sci USA 90(8), 3715-9.
Landegren, U., Kaiser, R., Sanders, J. & Hood, L. (1988). A ligase-mediated gene detection technique. Science 241(4869), 1077-80.
Leung, D. W., Chen, E. & Goeddel, D. V. (1989). A method for random mutagenesis of a defined DNA segment using a modified polymerase chain reaction. Technique 1, 11-15.
Lim, F. & Sun, A. M. (1980). Microencapsulated islets as bioartificial endocrine pancreas. Science 210(4472), 908-10.
Lissant, K., Ed. (1974). Emulsions and Emulsion Technology. Vol. 1-3. New York: Marcel Dekker.
Lizardi, P. M., Huang, X., Zhu, Z., Bray-Ward, P., Thomas, D. C. & Ward, D. C. (1998). Mutation detection and single-molecule counting using isothermal rolling-circle amplification. Nat Genet 19(3), 225-32.
Low, N. M., Holliger, P. H. & Winter, G. (1996). Mimicking somatic hypermutation: affinity maturation of antibodies displayed on bacteriophage using a bacterial mutator strain. J Mol Biol 260(3), 359-68.
Lowman, H. B., Bass, S. H., Simpson, N. & Wells, J. A. (1991). Selecting high-affinity binding proteins by monovalent phage display. Biochemistry 30(45), 10832-8.
Mattheakis, L. C., Bhatt, R. R. & Dower, W. J. (1994). An in vitro polysome display system for identifying ligands from very large peptide libraries. Proc Natl Acad Sci USA 91(19), 9022-6.
Minshull, J. & Sternner, W. P. (1999). Protein evolution by molecular breeding. Curr Opin Chem Biol 3(3), 284-90.
New, R. R. C., Ed. (1990). Liposomes: a practical approach. The practical appraoch series. Edited by Rickwood, D. & Hames, B. D. Oxford: Oxford University Press.
Nissim, A., Hoogenboom, H. R., Tomlinson, I. M., Flynn, G., Midgley, C., Lane, D. & Winter, G. (1994). Antibody fragments from a ‘single pot’ phage display library as immunochemical reagents. Embo J 13(3), 692-8.
Olsen, M. J., Stephens, D., Griffiths, D., Daugherty, P., Georgiou, G. & Iverson, B. L. (2000). Function-based isolation of novel enzymes from a large library. Nat Biotechnol 18(10), 1071-4.
Ostermeier, M., Shim, J. H. & Benkovic, S. J. (1999). A combinatorial approach to hybrid enzymes independent of DNA homology. Nat Biotechnol 17(12), 1205-9.
Parinley, S. F. & Smith, G. P. (1988). Antibody-selectable filamentous fd phage vectors: affinity purification of target genes. Gene 73(2), 305-18.
Pedersen, H., Holder, S., Sutherlin, D. P., Schwitter, U., King, D. S. & Schultz, P. G. (1998). A method for directed evolution and functional cloning of enzymes. Proc Natl Acad Sci USA 95(18), 10523-8.
Perelson, A. S. & Oster, G. F. (1979). Theoretical studies of clonal selection: minimal antibody repertoire size and reliability of self-non-self discrimination. J Theor Biol 81(4), 645-70.
Pirrung, M. C. & Huang, C. Y. (1996). A general method for the spatially defined immobilization of biomolecules on glass surfaces using “caged” biotin. Bioconjug Chem 7(3), 317-21.
Pollack, S. J., Jacobs, J. W. & Schultz, P. G. (1986). Selective chemical catalysis by an antibody. Science 234(4783), 1570-3.
Roberts, R. W. (1999). Totally in vitro protein selection using mRNA-protein fusions and ribosome display. Curr Opin Chem Biol 3(3), 268-73.
Roberts, R. W. & Ja, W. W. (1999). In vitro selection of nucleic acids and proteins: What are we learning? Curr Opin Struct Biol 9(4), 521-9.
Roberts, R. W. & Szostak, J. W. (1997). RNA-peptide fuisions for the in vitro selection of peptides and proteins. Proc Natl Acad Sci USA 94(23), 12297-302.
Saiki, R. K., Gelfand, D. H., Stoffel, S., Scharf, S. J., Higuchi, R., Horn, G. T., Mullis, K. B. & Erlich, H. A. (1988). Primer-directed enzymatic amplification of DNA with a thermostable DNA polymerase. Science 239(4839), 487-91.
Sambrook, J., Fritsch, E. & Maniatis, T. (1989). Molecular Cloning. 2nd edit, Cold Spring Harbor Laboratory Press, New York.
Sano, T., Smith, C. L. & Cantor, C. R. (1992). Immuno-Pcr-Very Sensitive Antigen-Detection by Means of Specific Antibody-DNA Conjugates. Science 258(5079), 120-122.
Schatz, P. J., Cull, M. G., Martin, E. L. & Gates, C. M. (1996). Screening of peptide libraries linked to lac repressor. Methods Enzymol 267, 171-91.
Schweitzer, B. & Kingsmore, S. (2001). Combining nucleic acid amplification and detection. Curr Opin Biotechnol 12(1), 21-7.
Schweitzer, B., Wiltshire, S., Lambert, J., O'Malley, S., Kukanskis, K., Zhu, Z. R., Kingsmore, S. F., Lizardi, P. M. & Ward, D. C. (2000). Inmunoassays with rolling circle DNA amplification: A versatile platform for ultrasensitive antigen detection. Proceedings of the National Academy of Sciences of the United States of America 97(18), 10113-10119.
Sherman, P. (1968). Emulsion science, Academic Press, London.
Smith, G. P. & Petrenko, V. A. (1997). Phage display. Chemical reviews 97(2), 391-410.
Stemmer, W. P. (1994a). DNA shuffling by random fragmentation and reassembly: in vitro recombination for molecular evolution. Proc Natl Acad Sci USA 91(22), 10747-51.
Stemmer, W. P. (1994b). Rapid evolution of a protein in vitro by DNA shuffling. Nature 370(6488), 389-91.
Sundberg, S. A., Barrett, R. W., Pirrung, M., Lu, A. L., Kiangsoontra, B. & Holmes, C. P. (1995). Spatially-addressable immobilisation of macromolecules on solid supports. J As Chem. Soc. 117, 12050-12057.
Szostak, J. W. & Wilson, D. S. (1999). In vitro selection of functional nucleic acids. Ann. Rev. Biochem. 68, 611-647.
Tawfik, D. S. & Griffiths, A. D. (1998). Man-made cell-like compartments for molecular evolution. Nat Biotechnol 16(7), 652-6.
Tramontano, A., Janda, K. D. & Lerner, R. A. (1986). Catalytic antibodies. Science 234(4783), 1566-70.
van Hal, D. A., Bouwstra, J. A. & Junginger, H. E. (1996). Nonionic surfactant vesicles containing estradiol for topical application. In Microencapsulation: methods and industrial applications (Benita, S., ed.), pp. 329-347. Marcel Dekker, New York.
Varga, J. M., Kalchschmnid, G., Klein, G. F. & Fritsch, P. (1991). Mechanism of allergic cross-reactions-I. Multispecific binding of ligands to a mouse monoclonal anti-DNP IgE antibody. Mol Immunol 28(6), 641-54.
Walker, G. T., Fraiser, M. S., Schram, J. L., Little, M. C., Nadeau, J. G. & Malinowski, D. P. (1992a). Strand displacement amplification—an isothermal, in vitro DNA amplification technique. Nucleic Acids Res 20(7), 1691-6.
Walker, G. T., Little, M. C., Nadeau, J. G. & Shank, D. D. (1992b). Isothermal in vitro amplification of DNA by a restriction enzyme/DNA polymerase system. Proc Natl Acad Sci USA 89(1), 392-6.
Wang, A. M., Doyle, M. V. & Mark, D. F. (1989). Quantitation of mRNA by the polymerase chain reaction. Proc Natl Acad Sci USA 86(24), 9717-21.
Whateley, T. L. (1996). Microcapsules: preparation by interfacial polymerisation and interfacial complexation and their applications. In Microencapsulation: methods and industrial applications (Benita, S., ed.), pp. 349-375. Marcel Dekker, New York.
Wu, D. Y. & Wallace, R. B. (1989). The ligation amplification reaction (LAR)—amplification of specific DNA sequences using sequential rounds of template-dependent ligation. Genomics 4(4), 560-9.
Yelamos, J., Klix, N., Goyenechea, B., Lozano, F., Chui, Y. L., Gonzalez, F. A., Pannell, R., Neuberger, M. S. & Milstein, C. (1995). Targeting of non-Ig sequences in place of the V segment by somatic hypenmutation. Nature 376(6537), 225-9.
Zaccolo, M., Williams, D. M., Brown, D. M. & Gherardi, E. (1996). An approach to random mutagenesis of DNA using mixtures of triphosphate derivatives of nucleoside analogues. J Mol Biol 255(4), 589-603.
Zhao, H., Giver, L., Shao, Z., Affholter, J. A. & Arnold, F. H. (1998). Molecular evolution by staggered extension process (StEP) in vitro recombination. Nat Biotechnol 16(3), 258-61.

1. A method for selecting one or more genetic elements encoding a gene product having a desired activity, which method comprises:
(i) providing a plurality of genetic elements comprising a nucleic acid optionally encoding a gene product;
(ii) optionally expressing the nucleic acids to produce the gene products, and allowing the desired activity of the nucleic acids or gene products to result, directly or indirectly, in the modification of the genetic elements which contained or encoded them;
(iii) associating one or more modulators of a nucleic acid replication system with a genetic element which has been modified by a gene product; and
(iv) selectively amplifying the nucleic acid component of those genetic elements which have been modified by the gene product.

2. A method for selectively amplifying nucleic acid sequences from a plurality of genetic elements encoding gene products, which method comprises
(i) providing a plurality of genetic elements comprising a nucleic acid optionally encoding a gene product;
(ii) optionally expressing the nucleic acids to produce the gene products, and allowing the desired activity of the nucleic acids or gene products to result, directly or indirectly, in the modification of the genetic elements which contained or encoded them;
(iii) selectively attaching to the modified genetic element at least a first component which potentiates amplification of the nucleic acid molecules;
(iv) dividing the plurality of genetic elements into a number of separate compartments;
(v) providing in each compartment further components for nucleic acid amplification such that only nucleic acid molecules linked to modified genetic elements to which said first component has been attached will be amplified; and
(vi) allowing amplification to occur in the compartments of the nucleic acid component of those genetic elements to which said first component has been attached.

3. A method for selectively amplifying nucleic acid sequences from a plurality of genetic elements encoding gene products, which method comprises
(i) providing a plurality of genetic elements comprising a nucleic acid optionally encoding a gene product;
(ii) optionally expressing the nucleic acids to produce the gene products, and allowing the desired activity of the nucleic acids or gene products to result, directly or indirectly, in the modification of the genetic elements which contained or encoded them;
(iii) selectively attaching to unmodified components of genetic elements at least a first component detrimental to the amplification of the nucleic acid molecules comprised by the genetic elements;
(iv) dividing the plurality of genetic elements into a number of separate compartments;
(v) providing in each compartment further components for nucleic acid amplification; and
(vi) allowing nucleic acid amplification to occur in the compartments.

4. A method according to claim 2 or claim 3 wherein substantially all of the compartments contain only one genetic element.

5. A method according to claim 1, 2 or 3 wherein the desired activity is a binding activity.

6. A method according to claim 1, 2 or 3 wherein the desired activity is the ability to convert a chemical substrate into product.

7. A method according to claim 6 wherein the desired activity is a catalytic activity.

8. A method according to claim 1, 2 or 3 wherein the desired activity is a regulatory activity.

9. A method according to claim 1, 2 or 3 wherein the expressed gene product becomes physically linked to the genetic element to form a complex.

10. A method according to claim 9 wherein the complexes are further reacted to induce a conditional change in the genetic element dependent on the presence of gene products with the desired activity in the complex.

11. A method according to claim 2 or 3 wherein the further components for nucleic acid amplification are selected from the group consisting of components for polymerase chain reaction (PCR), or a variant thereof, ligase chain reaction (LCR), strand displacement amplification (SDA), and nucleic acid sequence-based amplification (NASBA) or self-sustaining sequence replication (3SR), transcription-mediated amplification (TMA) and rolling circle amplification (RCA).

12. A method according to claim 11 wherein the first component comprises an oligonucleotide primer.

13. A method according to claim 11 wherein the first component comprises a nucleic acid polymerase.

14. A method according to claim 11, wherein the first component comprises a nucleic acid which will compete for amplification with the nucleic acid comprised in the genetic element.

15. A method according to claim 11, wherein the first component comprises a nucleic acid which shares PCR primer annealing sites in common with the nucleic acid comprised by the genetic element, such that said first component competes for amplification by PCR with the nucleic acid comprised by the genetic element.

16. A method according to claim 12 wherein, after nucleic acid replication, single-stranded nucleic acid products are removed.

17. A method according to claim 2 or 3 wherein the first component comprises a moiety capable of binding to a modified genetic element but not an unmodified genetic element.

18. A method according to claim 17 wherein the moiety is an antibody.

19. A method according to claim 1, 2 or 3 wherein step (ii) comprises the steps of:
(a) optionally expressing the genetic elements to produce genetic elements in which the expressed gene products become a component of the genetic element containing the genes that encoded them;
(b) compartmentalisation of the genetic elements into microcapsules; and
(c) allowing the desired activity of the gene or gene products to result, directly or indirectly, in the modification of the genetic elements which contained/encoded them.

20. A method according to claim 1, 2 or 3 wherein step (ii) comprises the steps of:
(a) compartmentalisation of genetic elements into microcapsules;
(b) expressing the genetic elements to produce their respective gene products within the microcapsules; and
(c) allowing the desired activity of the gene or gene products to result, directly or indirectly, in the modification of the genetic elements which contained/encoded them.

21. A method according to claim 1, 2 or 3 wherein compartmentalisation comprises emulsification into microcapsules by forming a water-in-oil emulsion with an aqueous phase comprising the genetic elements, bound first component and further components for amplification in an oil-based medium.

22. A method according to claim 1, 2 or 3 wherein the genetic element comprises the gene attached to a solid phase.

23. A method according to claim 22 wherein the solid phase is a microsphere.

24. A according to claim 1, 2 or 3 wherein the plurality of nucleic acid molecules is obtained from a library of nucleotide sequences encoding a gene product and variants thereof.

25. A method according to claim 1, 2 or 3 wherein the plurality of nucleic acid molecules is obtained from a library of nucleotide sequences and the number of nucleotide molecules in the plurality of nucleotide molecules is not significantly greater than the diversity of the library.

26. A method according to claim 24 wherein said library is a PCR-assembled combinatorial library.

27. A method according to claim 25 wherein said library is a PCR-assembled combinatorial library.

28. A method of in vitro evolution comprising the steps of:
(i) selecting one or more nucleic acids from a library of nucleic acids comprised in genetic elements according to any preceding claim;
(ii) mutating and/or recombining the selected nucleic acid(s) in order to generate a further library of nucleic acids, optionally encoding a repertoire of gene products;
(iii) iteratively repeating steps (i) and (ii) in order to obtain a gene or gene product with enhanced activity

29. A method according to claims 1, 2 or 3 which further comprises recovering one or more of the amplified nucleic acid molecules and determining all or part of their nucleotide sequence.

30. A kit comprising:
(i) a plurality of genetic element optionally encoding a plurality of gene products, each genetic element comprising a coding sequence operably linked to a regulatory sequence which is capable of directing expression of a gene product encoded by the sequence, wherein the desired activity of the gene or gene products results, directly or indirectly, in the modification of the genetic elements which contained/encoded them;
(ii) a first component required for amplification of the nucleic acid constructs which is capable of selectively binding to genetic elements modified by the action of the gene or gene products but not to unmodified genetic elements.

31. A kit according to claim 30 wherein the genetic element comprises the gene attached to a solid phase, such as a microsphere.

32. A method of selecting a nucleic acid with a desired activity or a nucleic acid encoding a gene product having a desired activity, which method comprises subjecting a plurality of nucleic acid molecules, optionally encoding a plurality of gene products, to selective amplification by the method of claims 1, 2, 3 or 28 and selecting one or more nucleic acid molecules which are amplified.

33. A method of selecting a variant of a gene or gene product which variant has altered activity compared with the gene or gene product, which method comprises subjecting a plurality of nucleic acid molecules and variants thereof, optionally encoding the gene product and variants thereof, to selective amplification by the method of claims 1, 2, 3 or 28 and selecting a nucleic acid molecule encoding a variant of the gene product which is amplified to a different extent to a nucleic acid molecule encoding the gene product.

34. A gene or gene variant selected by the method of claim 32.

35. A gene or gene variant selected by the method of claim 33.

36. A gene product or gene product variant selected by the method claim 32.

37. A gene product or gene product variant selected by the method claim 33.


SEQUENCE LISTING (9 sequences)

SEQ ID NO: 1 - DNA, 18 bp, Artificial
  Oligonucleotide primer aneealing upstream of T7 promoter of pIVEX vector
caggcgccattcgccatt

SEQ ID NO: 2 - DNA, 18 bp, Artificial
  Oligonucleotide primer annealing upstream of T7 promoter in pIVEX vector
ccagctggcgaaaggggg

SEQ ID NO: 3 - DNA, 18 bp, Artificial
  Oligonucleotide primer annealing upstream of T7 promoter in pIVEX vector
aagttgggtaacgccagg

SEQ ID NO: 4 - DNA, 18 bp, Artificial
  Oligonucleotide primer annealing upstream of T7 promoter in pIVEX vector
aagttgggtaacgccagg

SEQ ID NO: 5 - DNA, 17 bp, Artificial
  Oligonucleotide primer (pIVB series) annealing downstream of expressed gene
tatccggatatagttcc

SEQ ID NO: 6 - DNA, 17 bp, Artificial
  Oligonucleotide primer (pIVB series) annealing downstream of expressed gene
cacacccgtcctgtgga

SEQ ID NO: 7 - DNA, 17 bp, Artificial
  Oligonucleotide primer (pIVB series) annealing downstream of expressed gene
gtcgccatgatcgcgta

SEQ ID NO: 8 - DNA, 17 bp, Artificial
  Oligonucleotide primer (pIVB series) annealing downstream of expressed gene
cctgctcgcttcgctac

SEQ ID NO: 9 - DNA, 17 bp, Artificial
  Oligonucleotide primer (pIVB series) annealing downstream of expressed gene
agaaattgcatcaacgc